(19) United States
(12) Patent Application Publication
(10) Pub. No.: US 2007/0292967 A1
(43) Pub. Date: Dec. 20, 2007

(54) Assay for Ligands of the Ecdysone Receptor

(75) Inventors: Lloyd Douglas Graham, North Ryde (AU); Wynona Marguerite Johnson, Mount Waverly (AU); Marianne Bliese, Mount Waverly (AU); Garry Noel Hannan, Hunters Hill (AU); Ronald Johnston Hill, Forestville (AU)

Correspondence Address: GREENLEE WINNER AND SULLIVAN P C, 4875 PEARL EAST CIRCLE, SUITE 200, BOULDER, CO, 80301, US

(73) Assignee: COMMONWEALTH SCIENTIFIC AND INDUSTRIAL RESEARCH OR, Campbell, Australian Capital Territory (AU)

(21) Appl. No.: 10/596,154

(22) PCT Filed: Dec. 1, 2004
(86) PCT No.: PCT/AU04/01701

§ 371 Date: Apr. 9, 2007

(30) Foreign Application Priority Data
Dec. 1, 2003 (AU) 2003906637

(51) Int. Cl. G01N 33/53; C07J 21/00; C07J 17/00; A01N 25/00

(52) U.S. Cl. 436/501; 540/114; 540/115; 540/6; 552/541; 435/7.1; 424/405

(57) ABSTRACT

The present invention provides an ecdysteroid derivative in which a fluorescent moiety is attached to an ecdysteroid moiety by derivatisation of a hydroxyl group on the alkyl side chain of the ecdysteroid moiety. The derivative is capable of binding to an ecdysone receptor or ligand domain thereof and can be used in assays.

### FIELD OF THE INVENTION

The present invention relates to assays for ecdysone receptor ligands. Specifically, the invention provides fluorescent conjugates that retain the ability to bind ecdysone receptors and are functional as tracer ligands in fluorescence polarization (FP) assays comprising ecdysone receptors or the ligand-binding domain portions thereof. The invention is particularly useful for high-throughput screening of compound libraries with the aim of identifying lead compounds for pesticide development or novel effectors for ecdysone receptor gene switches.

### BACKGROUND OF THE INVENTION

In the 1960s, Carroll Williams pointed out that over 99% of insect species are either innocuous or beneficial from the human point of view. Some are even indispensable, e.g. bees via their role in pollination. Approximately 0.1% of insects are actually pests. Williams suggests that a new generation of safer insecticides exhibiting specificity for particular pests might be developed based on the chemistry of the insect's own hormones (Williams, 1967a, b). The levels of the non-peptide hormones controlling growth and development, 20-hydroxyecdysone (2β,3β,14α,20R,22R,25-hexahydroxy-5β-cholest-7-ene-6-one) and juvenile hormone, are precisely controlled. Inappropriate levels of compounds with ecdysteroid or juvenoid activity lead to major perturbation of insect development and subsequent lethality.

A problem with this approach, not initially appreciated, stems from the efficient mechanisms insects possess for clearing these hormones by metabolic degradation during normal development. This problem might be overcome by the discovery of compounds exhibiting high receptor affinities but with different chemistries to the natural hormones and thus not subject to the host's catabolic pathways.

The two non-peptide hormones known to play key roles in regulating insect growth and development are the steroid moulting hormone, 20-hydroxyecdysone, hereafter referred to as ecdysone, and the sesquiterpenoid juvenile hormone, hereafter referred to as JH. JH is responsible for maintaining larval or nymphal states in moulting insects in addition to a role in adults in the regulation of reproductive processes. The titre of ecdysone may rise and fall as many as six or more times during the life cycle of insects, regulating, for example, the moulting process between larval instars, the synthesis of new cuticle, the onset of metamorphosis (after a decline in JH titre) and aspects of vitellogenesis in the adult ovary. The giant polytene chromosomes seen in the dipteran Drosophila melanogaster, have given insights into the complexity of the response to a rise in ecdysone titre at the level of changes in gene expression. It was postulated by Ashburner and co-workers (Ashburner et al., 1974) that ecdysone exerts its action in regulating gene expression via a protein receptor. A few early responding genes produce further gene transcription regulatory proteins that transmit the response to a whole bank of late responding genes; these regulatory proteins can be detected in action at the late-responding chromosomal loci (Hill et al., 1993).

Over the past decade much progress has been made in understanding the molecular mechanisms underlying the key role of ecdysone in controlling insect development. This research has been led by studies involving the combined power of genetics and molecular biology employing the fly D. melanogaster. Of particular importance to the present application has been the elucidation of the nature of the ecdysone receptor. It has been shown to be a heterodimer made up of the products of two genes called ecr and usp (Yao et al., 1993). The protein products of these genes, EcR and USP, are members of the nuclear receptor superfamily. This family is characterised by an overall structural plan in which a series of domains impart, in order from the N-terminus: transcriptional activation, DNA binding, nuclear localisation and ligand binding. The ligand-binding domain (LBD) also imparts transactivation in response to the binding of agonist ligands. Both the EcR and USP subunits of ecdysone receptors have been cloned from a number of insects—see for example Koelle et al., 1991; Harman & Hill, 1997; Hannan & Hill 2001; Oro et al., 1990.

Until the 1980's, chemical approaches to the development of ecdysone mimics were hampered by the structural complexity and synthetic inaccessibility of the steroids for commercial-scale field applications. However in 1988, Rohm and Haas Company scientists (Wing et al., 1988; Wing, 1988) reported that a class of bisacylhydrazine insecticides, which the company had discovered serendipitously, were acting primarily via interaction with ecdysone receptors. The binding affinity of members of this class for an ecdysone receptor correlates well with the strength of their moulting hormone activity (Minakuchi et al., 2003). Members of this class display remarkable selectivity at the level of orders within the Insecta, for example RH-5992 is some two to three orders of magnitude more effective against Lepidotera than it is against Diptera. This difference correlates with different dissociation constants for interaction of the compounds with ecdysone receptors from the two insect orders (Dhadialla et al., 1998). Although subsequent studies (Sundaram et al., 1998) have demonstrated a contribution in some cases by active transport clearance, there is little doubt that variation in the structure of the ecdysone receptors per se between different orders plays a very significant role in underlying the selectivity of extant insecticides in this class.

The selectivity of the bisacylhydrazines for the Lepidoptera and some Colcoptera has both positive and negative connotations. On the positive side, we see a harbinger of safer, more environmentally-friendly insecticides targeting a receptor not only absent from vertebrates but also exhibiting sufficient variation across the Insecta to allow discrimination between pests and friendly or innocuous species. On the negative side, the present relatively narrow spectrum of activity limits sales and also leaves a significant number of insect orders that cannot be controlled by safe ecdysone receptor targeting chemistries. Industry has been trying to extend the spectrum of activity of agents with this mode of action but with relatively little success.

Biological assays to measure the activity of ecdysone receptor agonists and antagonists are well known in the field. Traditional screens for ecdysone receptor agonists examine candidate compounds for an ability to induce the moulting or pupation of insect larvae (Becker, 1941; Cymborowski, 1989), the evagination of imaginal discs (Fristrom & Yund, 1976) or morphological transformation of the Drosophila BII cell line (Clément et al., 1993). More recent assays use mammalian or other eukaryotic cells that have been co-transfected with a recombinant ecdysone receptor and a reporter gene linked to an appropriate response element. Both types of screen can also be reformatted to detect non-agonist ligands (antagonists), which can be recognised by their ability to inhibit the activation of the receptor by an agonist provided as a standard component of the assay (Yang et al., 1986; Oberdorster et al., 2001). In addition, there are in vitro binding assays in which intact insect cells, cell extracts or purified recombinant ecdysone receptors are incubated with a radioactive ecdysone receptor ligand such as [³H]ponasterone A. These assays detect both agonists and antagonists, because both types of ligand compete with the radioactive tracer for binding to the ecdysone receptor (Yund et al., 1978; Cherbas et al., 1988). Binding affinity and inhibitor potency may also be measured for candidate inhibitors using biosensor technology, although the throughput of this format would generally be limited.

The ability to perform high-throughput screening of compound libraries against selected ecdysone receptors (or the ligand-binding portions thereof) should aid in the discovery of novel ecdysone receptor agonists and antagonists. Where the receptor in question is the ecdysone receptor from a pest insect, some of the newly-identified ligands may be able to disrupt the normal development and maturation of the relevant pest, i.e. the compounds may serve as lead compounds for insecticide development.

Furthermore, since ecdysone receptors and their functional domains are employed as components of ecdysone switches for the control of reporter and therapeutic genes in mammalian cells (Lafont & Dinan, 2003; Yang et al., 1986) and for control of transgenes more generally in agriculturally important species, both animal and plant (Lafont & Dinan, 2003; Padidam et al., 2003), the ability to screen compound libraries against selected ecdysone receptors (or the ligand-binding portions thereof) should aid in the discovery of safer and/or more effective ligands to act as effectors for such switches.

The current barriers to large-scale screening of chemical libraries against ecdysone receptors are: (1) the lack of availability of purified ecdysone receptors from pest insect species, and (2) the need to use radioactivity-based assays that are expensive, require a capture/wash step that is difficult to automate, and are disfavoured on health, safety and environmental grounds.

In contrast to radioactivity-based assays, fluorescence-based assays do not require the synthesis, handling, or disposal of radioactive ligands, nor do they require the use of hazardous scintillant cocktails. They are therefore preferred from an occupational health and safety perspective and have lower environmental impact. Accordingly, the use of fluorescence-based assays is less encumbered by licensing and disposal regulations. In any case, they are usually much less expensive to operate than radioactive assays, and almost always give more rapid read-outs (Sportsman & Leytes, 2000). A particularly useful form of fluorescence-based assays relies upon the phenomenon of fluorescence anisotropy or fluorescence polarization (FP), where polarized light is used to excite a fluorophore-containing ligand. Only fluorophores parallel to the polarization plane absorb the light and become excited. During the lifetime of the resulting excited state, free ligand molecules rotate by molecular tumbling such that the polarization plane of the emitted light differs from that of the excitation beam. In constrast, ligands bound to large molecules (such as receptor proteins) will tumble much more slowly and, provided the excitation lifetime of the fluorophore is sufficiently short, the emitted light will be largely in the same plane as the excitation beam. To evaluate the polarization of an assay solution, two measurements are needed: the first using a polarized emission filter parallel to the excitation filter (S-plane), and the second using a polarized emission filter perpendicular to the excitation filter (P-plane). The overall fluorescence polarization value, given in mP (milli-Polarization level), is given by the equation
Polarization (mP)=1000(S−G.P)/(S+G.P)
where S and P are the S-plane and P-plane fluorescence count rates and G is an instrument and assay-dependent grating factor (Perkin Elmer Life Sciences, Application note for Victor² V multilabel counter, January 2000). The basic outcome is that the fluorescent ligand is small and, if free in solution, its rapid tumbling results in low mP values. A bound fluorescent ligand tumbles at the much slower speed of the macromolecule to which it is bound, resulting in high mP values. The observed mP value for each assay represents a weighted average of the signals from the bound and free ligand populations (Owicki, 2000; Prystay et al., 2001), and this value can be measured by instruments with the appropriate optics and computational software. Such instruments are readily available from laboratory instrument suppliers, and can be obtained in versions designed either for reading individual tubes or multiwell plates. With a fluorescence lifetime of around 4 ns, fluorescein is well suited to the rotation speeds of molecules in receptor-ligand binding assays (Owicki, 2000), and therefore most of the commercially available FP detectors are provided with the appropriate filter sets for this fluorophore.

FP assays have been developed to detect ligand binding to antibodies (Jiskoot et al., 1991), mammalian nuclear hormone receptors (Parker et al., 2000), G-protein coupled receptors (Prystay et al., 2001), and other macromolecules. FP assays have also been adapted to allow the measurement of enzyme activities (Checovich et al., 1995; Parker et al., 2000). A great advantage of FP assays is that there is no need for bound ligand to be separated from free ligand, and therefore FP assays do not require the receptor to be captured and washed (Checovich et al., 1995). Since such capture/wash steps are typically slow and difficult to automate, FP assays have become a preferred platform for high throughput screening whenever an appropriate ligand is available (Wedin, 1999). Compared to other fluorescence-based techniques, FP assays are relatively insensitive to changes in fluorescence intensity such as those that might arise as a result of quenching by absorbance due to library compounds (Sportsman & Leytes, 2000). Moreover, they can be used with turbid or even opaque assay mixtures, such as those containing poorly soluble test compounds (Checovich et al., 1995). As an additional benefit, FP is inherently suitable for miniaturization, and has been demonstrated to work in assays with final volumes as small as 4 μl (Sportsman & Leytes, 2000).

Three-dimensional X-ray diffraction studies have shown that, when an ecdysteroid is bound to an ecdysone receptor LBD, it is fully enclosed within a binding pocket formed by the protein. These studies are the subject of International Patent Application No. PCT/AU2004/00713 which is incorporated herein by reference. Surprisingly, the present inventors have found that fluorophores can be attached to the steroid molecule, preferably on the steroid side chain, with no detrimental effect on the ability of the ecdysteroid to bind to ecdysone receptors. The present inventors have also demonstrated that such ecdysteroid-fluorophore conjugates can be used in conjunction with recombinant ecdysteroid receptor protein subsegments as the basis for a fluorescence polarisation assay/screen for ligands for ecdysone receptors. These matters provide the subject of the present invention.

### SUMMARY OF THE INVENTION

Compounds with a fluorescent tag and their method of preparation and use are described. These compounds are useful as ligands in in vitro ligand binding assays, and, in particular, in fluorescence polarization (FP) assays for ecdysone receptor ligands. The present invention also provides assays for screening compounds for their ability to interact with ecdysone receptors.

In a first aspect the present invention provides a compound selected from the group of compounds consisting of general structures 1a, 2a, and 3a which interact with an ecdysone receptor or ligand binding domain (LBD) thereof; 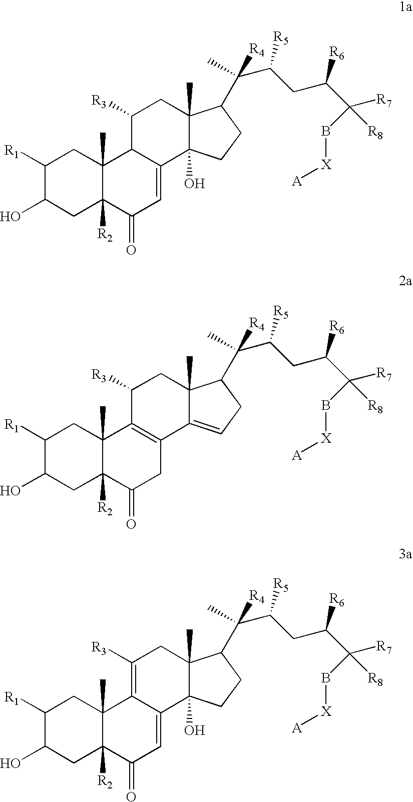
wherein B is CH₂O, CH₂S, CH₂NH, O, S, or NH; X is a linking group; A is a fluorescent moiety; R¹-R⁵ are independently selected from H, alkyl, haloalkyl, OH, or halogen, R⁷-R⁸ are independently selected from H, alkyl, haloalkyl, OH, or halogen or R⁷ and R⁸ together are ═CH₂; R⁶ is selected from H, OH, alkyl, ═CH₂ or halogen, or R⁶ together with R⁷ is a double bond.

Preferably R¹, and R⁵ are OH; R² is H or OH; R³ is H; R⁴ is H or OH; R⁶ is selected from H, OH, CH₃, CH₃CH₂, or (CH₃)₂ CH; R⁷ and R⁸ are independently selected from H, OH, CH₃, or R⁷ and R⁸ together can be ═CH₂; R⁶ together with R⁷ can be a double bond.

In a second aspect, the present invention provides a compound selected from the group of compounds consisting of general structures 1b, 2b, and 3b which interact with an ecdysone receptor or ligand binding domain (LBD) thereof; 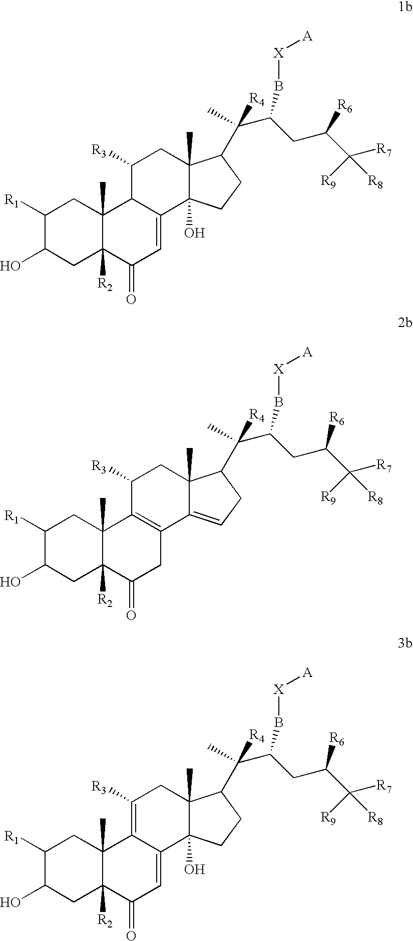
wherein B is CH₂O, CH₂S, CH₂NH, O, S, or NH; X is a linking group; A is a fluorescent moiety; R¹-R⁴, R⁷-R⁹ are independently selected from H, alkyl, haloalkyl, OH, or halogen; R⁶ is selected from H, OH, alkyl, ═CH₂ or halogen.

Preferably R¹ and R⁴ are OH; R² and R³ are independently selected from H or OH; R⁶ is selected from H or CH₃, and R⁷, R⁸ and R⁹ are independently selected from the group H, OH, CH₃F, and I.

In a third aspect the present invention provides a compound which is an ecdysteroid mimic wherein the compound comprises a non-ecdysteroid moiety that interacts with an ecdysone receptor or ligand binding domain thereof, and wherein the compound further comprises a fluorescent moiety.

In a fourth aspect, the present invention provides a method for screening a candidate compound for its ability to interact with an ecdysone receptor or ligand binding domain (LBD) thereof in a competitive inhibition format, the method comprising the steps of:
(a) incubating with an ecdysone receptor or LBD thereof, a candidate compound and the compound, mimic or derivative of the invention; and
(b) measuring the level of binding of the compound, mimic or derivative of the invention to the ecdysone receptor or LBD thereof.

In a fifth aspect, the present invention provides an insecticidal compound identified by the assay according to the fourth aspect of the invention.

In a sixth aspect, the present invention provides an effector compound for ecdysone receptor gene switches, the compound being identified by the assay according to the fourth aspect of the invention.

In another aspect the present invention provides an ecdysteroid derivative wherein a fluorescent moiety is attached to an ecdysteroid moiety by derivatisation of a hydroxyl group on the alkyl side chain of the ecdysteroid moiety, wherein the derivative is capable of binding to an ecdysone receptor or ligand binding domain thereof.

### BRIEF DESCRIPTION OF THE FIGURES

FIG. 1 An analysis of freshly-prepared recombinant ecdysone receptor samples by 12% SDS-PAGE, with staining by Coomassic Blue. The sample lanes show representative immobilised metal-ion affinity chromatography (IMAC) eluates for receptors from three insect species, as follows: Lc, recombinant LBD heterodimer of ecdysone receptor from Lucilia cuprina (LcLBD); Mp, recombinant LBD heterodimer of ecdysone receptor from Myzus persicae (MpLBD); Bt, recombinant LBD heterodimer of ecdysone receptor from Bernisin tabaci (BtLBD). The receptor samples (8-12 μg protein per lane) were boiled in the presence of 5% (v/v) 2-mercaptoethanol before loading. In each receptor lane, the upper band of the major doublet is recombinant EcR subunit and the lower band is the recombinant USP subunit, while the additional faint bands are protein contaminants (readily visible due to the high protein load per lane). M: marker proteins, with molecular masses shown in kilodaltons (kDa) to the left.

FIG. 2 Inokosterone and its fluorescent conjugates (MB4603, MB4592, and MB4628) were tested for the ability to compete with [³H]ponasterone A for binding to Myzus persicae MpLBD. The Y-axis shows the actual amount of receptor-bound [³H]ponasterone A as a percentage of the maximum possible [³H]ponasterone A binding; the X-axis indicates the concentration of the competing ligand (inokosterone or fluorescent conjugate thereof). Abbreviations: Inoko, inokosterone. This figure shows that all three of the fluorescent conjugates of inokosterone were inimpaired relative to inokosterone itself in their ability to bind to the recombinant ecdysone receptor.

FIG. 3 A. Fluorescence polarization (FP) titration curves for the inokosterone-fluorescein conjugate MB4628 (36 nM) and recombinant ecdysone receptors. The concentrations of the latter are indicated by the X-axis, while the Y-axis shows final polarization values (mP) for the assays at equilibrium. Values around 100 mP indicate that all of the MB4628 is free; as the proportion of receptor-bound MB4628 increases, the mP value increases from this baseline in a sigmoid fashion. In terms of FP assays, a dynamic range of 235 mP, such as that observed here with MpLBD, is considered to be excellent. Abbreviations: Mp, MpLBD; Bt4, BtLBD; Lc, LcLBD; HaDEF, recombinant LBD heterodimer of ecdysone receptor from Helicoverpa armigera (HaLBD).

B. The effect of including the non-denaturing detergent CHAPS (3-[(3-cholamidopropyl) dimethylammonio]-1-propanesulphonate) in the FP titration of BtLBD with MB4628. CHAPS was either omitted (filled circles) or included at 2 mM (open circles) in assays containing 36 nM MB4628, while the concentrations of the functional BtLBD are indicated by the X-axis. For the CHAPS-free assays, the FP plate-reader was first standardised to read 100 mP for a solution of 36 nM MB4628 in FP assay buffer, whereas for CHAPS-containing assays it was standardised to read 100 mP for a solution containing 36 nM MB4628 and 2 mM CHAPS in FP assay buffer. The Y-axis shows final polarization values (mP) for the assays at equilibrium. Comparison of the titration curves shows that the presence of CHAPS greatly increased the dynamic range of the BtLBD FP assay. In terms of FP assays, the expanded range (260 mP) is considered to be excellent.

FIG. 4 The binding efficacy of well-known ecdysteroids assessed using the FP assay in a competitive inhibition format. For both MpLBD and BtLBD, the FP competitive inhibition assays ranked the binding affinity of these reference ligands as ponasterone A>muristerone A>20-hydroxyecdysone, the same order as that given by the conventional radioligand-based assay.

A. Assays were conducted by incubating 5 nM functional MpLBD with 36 nM MB4628 in the presence of increasing concentrations of the non-fluorescent reference ecdysteroids (20-OH-Ec, 20-hydroxyecdysone; MurA, muristerone A; PonA, ponasterone A). The concentrations of the latter are indicated by the X-axis, while the Y-axis shows final polarization values (mP) for the assays at equilibrium. The plot also shows (as solid lines) the upper and lower boundaries (mPₘₐₓ and mPₘᵢₙ, respectively; the placement of the latter is explained in the text) that were used to determine the position of the titration midpoint (dotted line).

B. Assays were conducted by incubating 10 nM functional BtLBD with 36 nM MB4628 and 2 mM CHAPS in the presence of increasing concentrations of the non-fluorescent reference ecdysteroids. The abbreviations, axes, and boundary lines are as described in part A.

FIG. 5 Relationship between the FP-derived Kᵢ values for each of the reference ecdysteroids and the corresponding Kᵢ values derived from the radioligand-based assay. The FP-derived Kᵢ value for each of the reference ecdysteroids, 20-hydroxyecdysone, muristerone A and ponasterone A, was plotted against the corresponding Kᵢ value from the radioligand-based assay. It is clear that the FP assay ranks the competitor ligands correctly in terms of potency, and that it displays increased powers of discrimination over the radioligand-based assay. The plot is based on Kᵢ values derived from MpLBD FP and radioligand-based competitive inhibition titrations (both done without CHAPS), BtLBD FP competitive inhibition titrations (done with 2 mM CHAPS), and BtLBD radioligand-based competitive inhibition titrations (done without CHAPS).

### DETAILED DESCRIPTION OF THE INVENTION

The invention provides fluorescent conjugates that are useful as ligands in vitro ligand binding assays, in particular, fluorescence polarization (FP) assays for ecdysone receptor ligands. The FP format is homogenous, ie., the binding reaction and FP measurement of each assay is performed in the same compartment (e.g. a single well in a multiwell plate). The assay is therefore ideally suited to the miniaturization and automation that underpins industrial high throughput screening programs. The fluorescent compounds can, for example, be prepared by reacting a reactive group in the fluorescent moiety with a nucleophilic group in the compound that binds to the ecdysone receptor.

Accordingly, in a first aspect, the present invention provides a compound selected from the group of compounds consisting of general structures 1a, 2a, and 3a which interact with an ecdysone receptor or ligand binding domain (LBD) thereof; 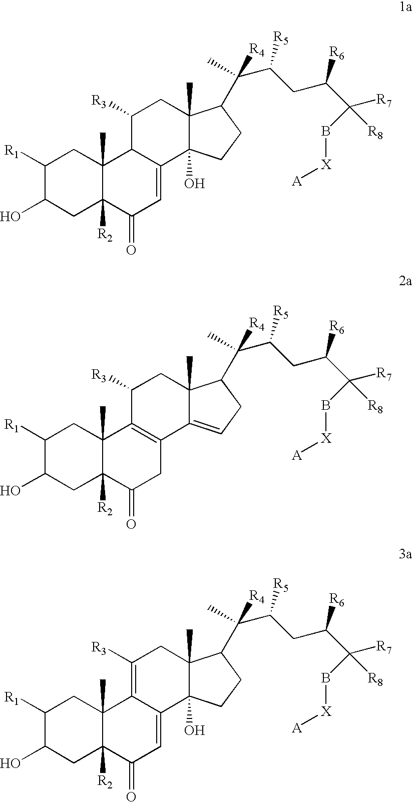
wherein B is CH₂O, CH₂S, CH₂NH, O, S, or NH; X is a linking group; A is a fluorescent moiety; R¹-R⁶ are independently selected from H, alkyl, haloalkyl, OH, or halogen, R⁷-R⁸ are independently selected from H, alkyl, haloalkyl, OH, or halogen or R⁷ and R⁸ together are ═CH₂; R⁶ is selected from H, OH, alkyl, ═CH₂ or halogen, or R⁶ together with R⁷ is a double bond.

It is preferred that that the alkyl groups are C1 to C20. In regard to R¹ and R² it is preferred that the alkyl group is C1 to C5.

In a preferred embodiment R¹, and R⁵ are OH; R² is H or OH; R³ is H; R⁴ is H or OH; R⁶ is selected from H, OH, CH₃, CH₃CH₂ or (CH₃)₂ CH; R⁷ and R⁸ are independently selected from H, OH, CH₃, or R⁷ and R⁸ together can be ═CH₂, R⁶ together with R⁷ can be a double bond.

It is preferred that X is selected from the group consisting of C(O)NH, C(S)NH, SO₂ and C(O). It is also preferred that B is CH₂O or O.

Preferably A is selected from the group consisting of unsubstituted and substituted fluorescein moieties, unsubstituted and substituted dansyl moieties, and unsubstituted and substituted coumarin moieties.

Preferably, the present invention provides a compound of general structure 1a wherein R¹ and R⁵ are OH, R³ is H, and R⁷ is CH₃, and B is CH₂O and X is selected from the group consisting of C(O)NH, C(S)NH, SO₂ and C(O).

It is further preferred that R², R⁴, and R⁸ are independently selected from H, alkyl, OH, or halogen; R⁶ is selected from H, OH, alkyl, ═CH₂ or halogen.

Preferably, R² is H, R⁴ is OH, R^{6 is H and R}⁸ is H.

In another preferred embodiment Preferably R¹, R⁴ and R⁵ are OH, R² is H or OH, R³ is H, R⁶ is H or CH₃, and R⁷ and R⁸ are CH₃.

It is further preferred that the fluorescent moiety is selected from the group consisting of unsubstituted and substituted fluorescein moieties, unsubstituted and substituted dansyl moieties, and unsubstituted and substituted coumarin moieties.

The fluorescent moiety may be attached by derivatisation of a hydroxyl group on the alkyl side chain of an ecdysteroid moiety that is capable of binding to an ecdysone receptor or ligand binding domain thereof. More preferably, fluorescent moiety is attached to the ecdysteroid by derivatisation of a reactive primary hydroxyl group on C-26 such as occurs in inokosterone, 26-hydroxyecdysone, 20,26-dihydroxyecdysone, makisterone B, amarasterone A, amarasterone B, ajugasterone B, sidasterone A, sidasterone B and 26-hydroxy-polypodine B.

In an alternative embodiment the fluorescent moiety is attached by derivatisation of a hydroxyl group at C-25 of an ecdysteroid selected from the group consisting of 20-hydroxyecdysone, makisterone A, polypodine B and rapisterone D.

Compounds of the invention having structures 1a, 2a, and 3a in which B is O may be prepared by standard synthetic procedures; for example see Odinokov et al. (2003) and Pis et al. (1994).

In a further preferred embodiment of the first aspect, the compound is selected from the group consisting of:

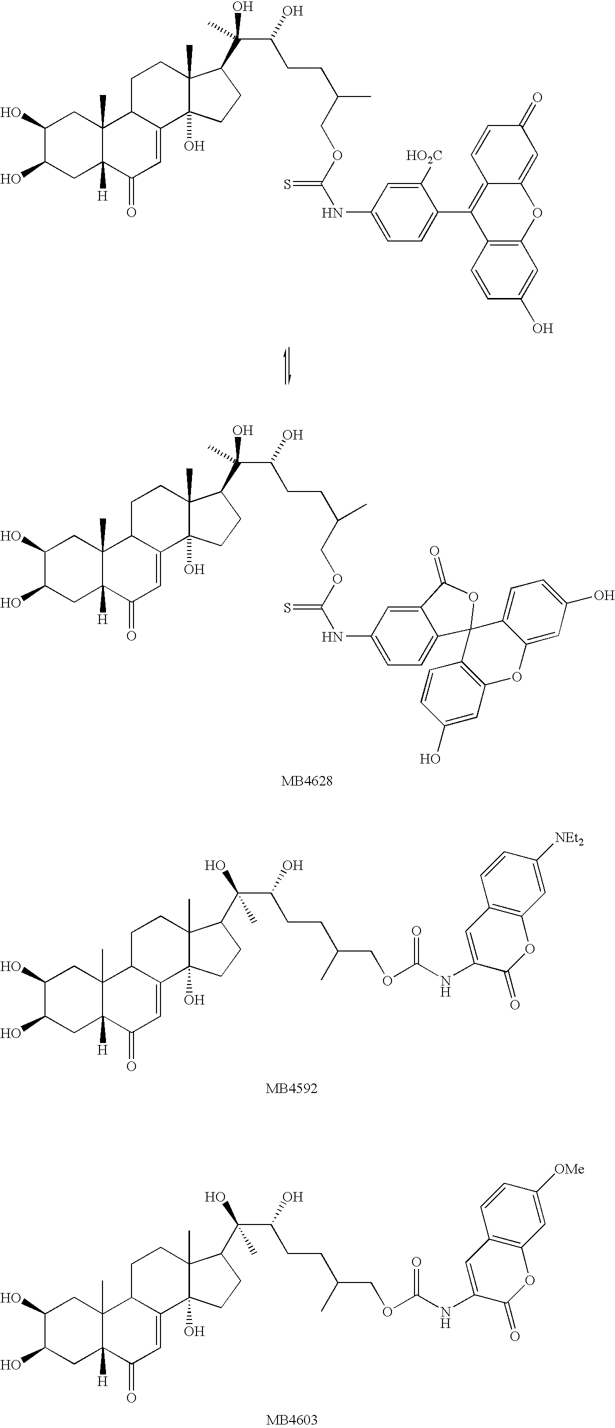

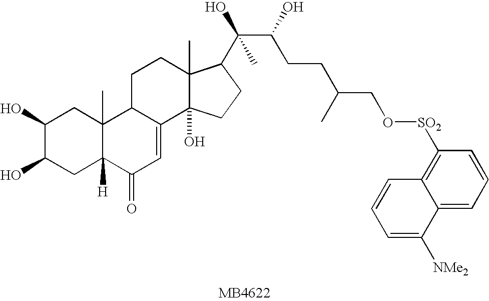

The structure of MB4628 may adopt different forms as shown above depending on the pH of the solution.

In a further embodiment of the present invention, the fluorescent moiety may be attached via a hydroxyl at an alternative position on the steroid side chain, such as a 22-OH, to give compounds of general structures 1b, 2b, and 3b.

Accordingly, in a second aspect, the present invention provides a compound selected from the group of compounds consisting of general structures 1b, 2b, and 3b which interact with an ecdysone receptor or ligand binding domain (LBD) thereof; 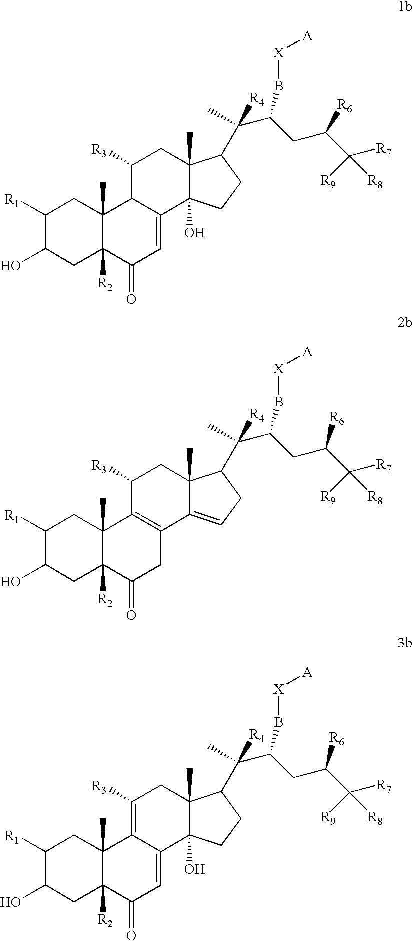
wherein B is CH₂O, CH₂S, CH₂NH, O, S, or NH; X is a linking group; A is a fluorescent moiety; R¹-R⁴, R⁷-R⁹ are independently selected from H, alkyl, haloalkyl, OH, or halogen; R⁶ is selected from H, OH, alkyl, ═CH₂ or halogen.

It is preferred that that the alkyl groups are C1 to C20. In regard to R¹ and R² it is preferred that the alkyl group is C1 to C5.

Preferably, X is selected from the group consisting of C(O)NH, C(S)NH, SO₂, and C(O).

Preferably, the fluorescent moiety is selected from the group consisting of unsubstituted and substituted fluorescein moieties, unsubstituted and substituted dansyl moieties, and unsubstituted and substituted coumarin moieties.

Preferably R¹ and R⁴ are OH; R² and R³ are independently selected from H or OH; R⁶ is selected from H or CH₃, and R⁷, R⁸ and R⁹ are independently selected from the group H, OH, CH₃, F, and I.

More preferably the fluorescent moiety is attached by derivatisation of a hydroxyl group at C-22 of an ecdysteroid selected from the group consisting of ponasterone A, 20-hydroxyecdysone, muristerone A, makisterone A, polypodine B, rapisterone D, 2β,3β,20R,22R-tetrahydroxy-25-fluoro-5β-cholest-8,14-dien-6-one, 5-deoxykaladasterone, 26-iodoponasterone A, and 25-fluoroponasterone A.

Compounds of the invention having structures 1b, 2b, and 3b where B is oxygen, may be prepared by standard synthetic procedures; for example, see Suksamram et al. (1995) and Suksamram et al. (2002).

It will be readily apparent to those skilled in the art that a number of the compounds of the present invention exist in both the 25R and 25S isomeric forms. The present invention is intended to cover both the separated 25R and 25S forms as well as mixtures thereof.

In another aspect the present invention provides an ecdysteroid derivative wherein a fluorescent moiety is attached to ecdysteroid moiety by derivatisation of a hydroxyl group on the alkyl side chain of the ecdysteroid moiety, wherein the derivative is capable of binding to an ecdysone receptor or ligand binding domain thereof.

Preferably the ecdysteroid derivative has general structure 1a, 2a, or 3a; 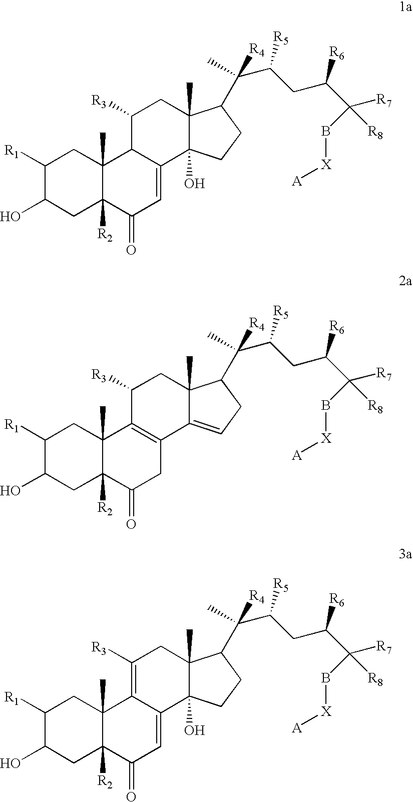
wherein B is CH₂O, CH₂S, CH₂NH, O, S, or NH; X is a linking group; A is a fluorescent moiety; R¹-R⁵ are independently selected from H, alkyl, haloalkyl, OH, or halogen, R⁷-R⁸ are independently selected from H, alkyl, haloalkyl, OH, or halogen or R⁷ and R⁸ together are ═CH₂; R⁶ is selected from H, OH, alkyl, ═CH₂ or halogen, or R⁶ together with R⁷ is a double bond.

In another embodiment the derivative has general structure 1b, 2b, or 3b; 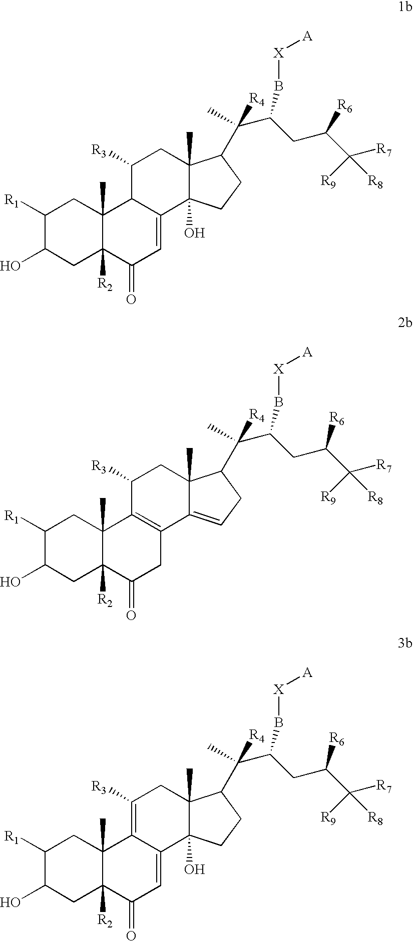
wherein B is CH₂O, CH₂S, CH₂NH, O, S, or NH; X is a linking group; A is a fluorescent moiety; R¹-R⁴, R⁷-R⁹ are independently selected from H, alkyl, haloalkyl, OH, or halogen; R⁶ is selected from H, OH, alkyl, ═CH₂ or halogen.

It is preferred that that the alkyl groups are C1 to C20. In regard to R¹ and R² it is preferred that the alkyl group is C1 to C5.

It is preferred that X is selected from the group consisting of C(O)NH, C(S)NH, SO₂ and C(O), and B is CH₂O or O.

It is further preferred that the fluorescent moiety is selected from the group consisting of unsubstituted and substituted fluorescein moieties, unsubstituted and substituted dansyl moieties, and unsubstituted and substituted coumarin moieties.

Where the derivative is of general structure 1a, 2a, 3a it is preferred that R¹, and R⁵ are OH; R² is H or OH; R³ is H; R⁴ is H or OH; R⁶ is selected from H, OH, CH₃, CH₃CH₂ or (CH₃)₂ CH; R⁷ and R⁸ are independently selected from H, OH, CH₃, or R⁷ and R⁸ together can be ═CH₂, R⁶ together with R⁷ can be a double bond.

Where the derivative is of general structure 1a, it is preferred that R¹ and R⁵ are OH, R³ is H, R⁷ is CH₃, and B is CH₂O and X is selected from the group consisting of C(O)NH, C(S)NH, SO₂ and C(O). It is also preferred that R², R⁴, and R⁸ are independently selected from H, alkyl, OH, or halogen; R⁶ is selected from H, OH, alkyl, ═CH₂ or halogen, more preferably R² is H, R⁴ is H and R⁸ is H.

In another embodiment where the derivative is of general structure 1a, 2a, 3a it is preferred that R¹, R⁴ and R⁵ are OH, R² is H or OH, R⁶ is H or CH₃, and R⁷ and R⁸ are H or CH₃.

Where the derivative is of general structure 1a, 2a, or 3a it is preferred that the ecdysteroid moiety is selected from the group consisting of inokosterone, 26-hydroxyecdysone, 20,26-dihydroxyecdysone, makisterone B, amarasterone A, amarasterone B, ajugasterone B, sidasterone A, sidasterone B, 26-hydroxy-polypodine B, 20-hydroxyecdysone, makisterone A, polypodine B and rapisterone D.

In a particularly preferred embodiment the derivative is selected from the group consisting of:

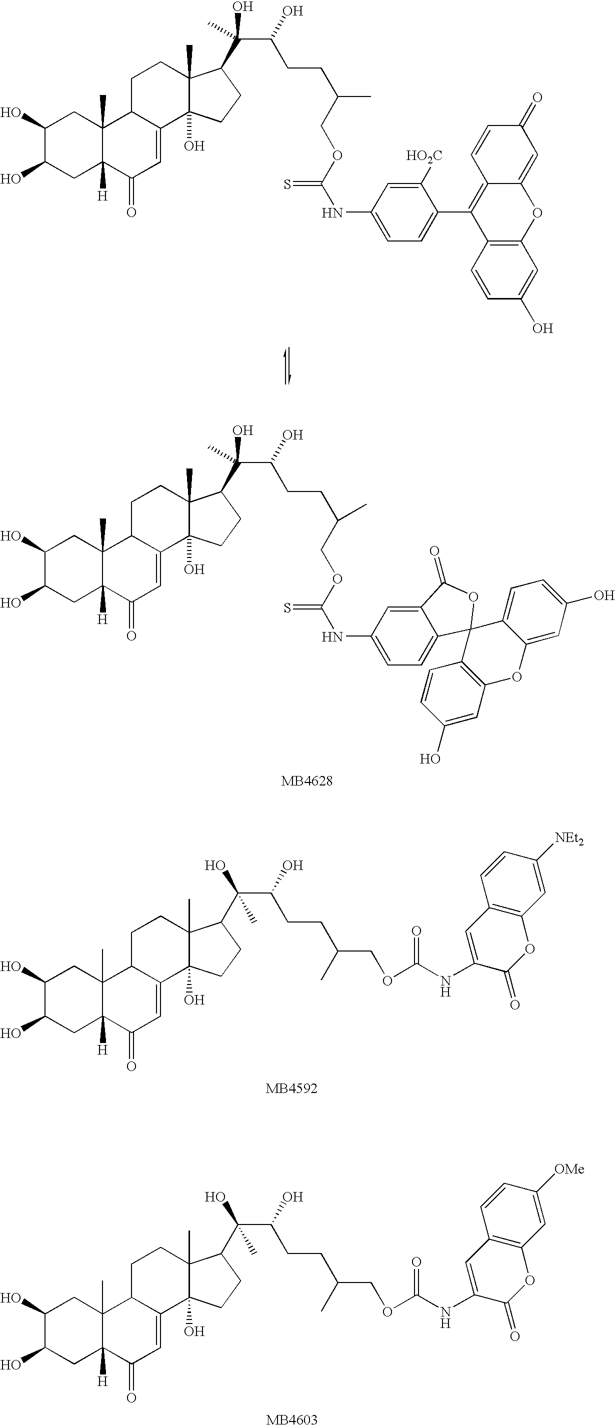

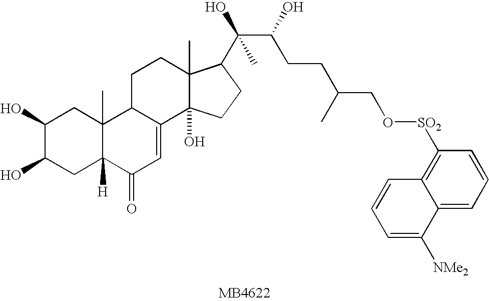

Where the derivative is of general structure 1b, 2b or 3b it is preferred that R¹ and R⁴ are OH; R² and R³ are independently selected from H or OH; R⁶ is selected from H or CH₃, and R⁷, R⁸ and R⁹ are independently selected from the group H, OH, CH₃, F and I.

Where the derivative is of general structure 1b, 2b or 3b it is also preferred that the ecdysteroid moiety is selected from the group consisting of ponasterone A, 20-hydroxyecdysone, muristerone A, makisterone A, polypodine B, rapisterone D, 2β,3β,20R,22R-tetrahydroxy-25-fluoro-5β-cholest-8,14-dien-6-one, 5-deoxykaladasterone, 26-iodoponasterone A, and 25-fluoroponasterone A.

It will be appreciated that the range of chromophores gives flexibility in the wavelength of observation for ligand-binding assays in which the fluorescent compound binds to a receptor protein or LBD thereof. Preferably, the fluorescent moiety is selected from the group consisting of unsubstituted and substituted fluorescein moieties, unsubstituted and substituted dansyl moieties, unsubstituted and substituted coumarin moieties. However, it will be apparent to those skilled in the art that many other fluorphores could be employed such as substituted and unsubstituted forms of the following: Cy5, Cy7, R-Phycoerythrin, Rhodamine, Texas Red, Alexa Fluors. Examples include the MoBiTec MFP series, e.g. MFP488, MFP555, MFP590 & MFP631; the Molecular Probes Oregon Green series, e.g. Oregon Green 488, Oregon Green 500, Oregon Green 514; the Molecular Probes Blue Series, e.g. Marina Blue, Pacific Blue, Cascade blue; Fluorescein derivatives, including Molecular Probes dyes such as FAM (e.g. 5-FAM, 6-FAM, 5(6)-FAM); jOE (e.g. 6-JOE), TET, HEX; Rhodamine derivatives e.g. Molecular Probes Rhodamine Green (a non-sulfonated version of Alexa Fluor 488), tetramethylrhodmaines (e.g. 5-TRITC, 6-TRITC, 5(6)-TRITC, 6-TAMRA), ROX (e.g. 6-ROX), Rhodamine 6G dyes (e.g. 5-CR 6G, 6-CR 6G, 5(6)-CR 6G, Rhodamine Red dyes; Bimane and its derivatives; the Molecular Probes BODIPY series, e.g. BODIPY FL, BODIPY TMR, BODIPY TR-X, BODIPY 530/550; various Molecular Probes Coumarin derivatives, e.g. AMCA-X, DMACA; and other fluorescent moieties listed in Table 1 (derived from the Salk Institute website) which is herein incorporated by reference. More preferably, the fluorescent moiety is fluorescein or a substituted fluorescein moiety.

TABLE 1
This is a table of some characteristics of known fluorochromes,
as presented on the Salk Institute website.

| | | | | |
|---|---|---|---|---|
| Probe | Ex (nm) | Em (nm) | MW | Notes |

Reactive and conjugated probes

| | | | | |
|---|---|---|---|---|
| Hydroxycoumarin | 325 | 386 | 331 | Reagent = Succinimidyl ester |
| Aminocoumarin | 350 | 445 | 330 | Reagent = Succinimidyl ester |
| Methoxycoumarin | 360 | 410 | 317 | Reagent = Succinimidyl ester |
| Cascade Blue | 375; 400 | 423 | 596 | Reagent = IIydrazide |
| Lucifer yellow | 425 | 528 | | |
| NBD | 466 | 539 | 294 | Molecular Probes NBD-X; |
| | | | | substituted nitrobenz-2-oxa- |
| | | | | 1,3-diazole compounds |
| R-Phycoerythrin | 480; 565 | 578 | 240 k | |
| (PE) | | | | |
| PE-Cy5 conjugates | 480; 565; 650 | 670 | | aka Cychrome, R670, Tri-Color, |
| | | | | Quantum Red |
| PE-Cy7 conjugates | 480; 565; 743 | 767 | | |
| APC-Cy7 | 650; 755 | 767 | | PharRed |
| conjugates | | | | |
| Red 613 | 480; 565 | 613 | | PE-Texas Red |
| Fluorescein | 495 | 519 | 389 | Reagent = FITC; pH sensitive |
| FluorX | 494 | 520 | 587 | (AP Biotech) |
| BODIPY-FL | 503 | 512 | | |
| TRITC | 547 | 572 | 444 | TRITC |
| X-Rhodamine | 570 | 576 | 548 | XRITC |
| Lissamine | 570 | 590 | | |
| Rhodamine B | | | | |
| Texas Red | 589 | 615 | 625 | Reagent = Sulfonyl chloride |
| Allophycocyanin | 650 | 660 | 104 k | |
| (APC) | | | | |
| Alexa Fluor 350 | 346 | 445 | 410 | (Molecular Probes) |
| Alexa Fluor 430 | 430 | 545 | 701 | (Molecular Probes) |
| Alexa Fluor 488 | 494 | 517 | 643 | (Molecular Probes) |
| Alexa Fluor 532 | 530 | 555 | 724 | (Molecular Probes) |
| Alexa Fluor 546 | 556 | 573 | 1079 | (Molecular Probes) |
| Alexa Fluor 555 | 556 | 573 | 1250 | (Molecular Probes) |
| Alexa Fluor 568 | 578 | 603 | 792 | (Molecular Probes) |
| Alexa Fluor 594 | 590 | 617 | 820 | (Molecular Probes) |
| Alexa Fluor 633 | 621 | 639 | 1200 | (Molecular Probes) |
| Alexa Fluor 647 | 650 | 668 | 1250 | (Molecular Probes) |
| Alexa Fluor 660 | 663 | 690 | 1100 | (Molecular Probes) |
| Alexa Fluor 680 | 679 | 702 | 1150 | (Molecular Probes) |
| Alexa Fluor 700 | 696 | 719 | | (Molecular Probes) |
| Alexa Fluor 750 | 752 | 779 | | (Molecular Probes) |
| Cy2 | 489 | 506 | 714 | (AP Biotech) |
| Cy3 | (512); 550 | 570; (615) | 767 | (AP Biotech) |
| Cy3.5 | 581 | 596; (640) | 1102 | (AP Biotech) |
| Cy5 | (625); 650 | 670 | 792 | (AP Biotech) |
| Cy5.5 | 675 | 694 | 1128 | (AP Biotech) |
| Cy7 | 743 | 767 | 818 | (AP Biotech) |

Legend:
Ex: Peak excitation wavelength (nm)
Em: Peak emission wavelength (nm)
MW: Molecular weight

It will be readily apparent to those skilled in the art that a number of the compounds of this embodiment of the present invention exist in both the 25R and 25S isomeric forms. The present invention is intended to cover both the separated 25R and 25S forms as well as mixtures thereof.

By “ecdysone receptor” we mean the full length, functional EcR/USP heterodimeric receptor. It will be appreciated that compounds may bind to the receptor in a number of ways that affect receptor function, for example (a) binding to the EcR receptor subunit alone, (b) binding to the USP receptor subunit alone, and (c) binding to the region of the receptor that effects heterodimerisation of the receptor subunits.

By “ligand binding domain” (LBD) we mean the region of the functional receptor that binds ecdysteroid. Typically, this is a region of the EcR subunit that contains the ecdysteroid binding pocket and is presented as a heterodimer with the corresponding region of the USP subunit. Thus, the LBD is typically a portion of the full-length functional receptor and comprises fragments of the full-length receptor subunits, EcR and USP.

It would readily be apparent to a person skilled in the art, however, that the fluorescent compound may be based on other members of the ecdysteroid family. Numerous derivatives of 20-hydroxyecdysone have been isolated and identified from plant and animal sources. A non-limiting list of such ecdysteroids is provided in Tables 1 and 2 of Horn and Bergamasco (1985) which is incorporated herein by reference. A non-limiting list of ecdysteroids, together with an indication of their binding efficacies for the ecdysone receptor of Drosophila, is provided in Tables 1 and 2 of Dinan et al. (1999) which is also incorporated herein by reference.

The present inventors have shown that compounds that conform to the general structure 1a, such as MB4628, MB4592, MB4603 and MB4622, are suitable for use in fluorescence polarization assays comprising ecdysone receptors or the LBD portion thereof. Since the fluorescent moiety in MB4628 is fluorescein, one of the most commonly used fluors in academia and industry, the relevant fiber sets for measuring its fluorescence intensity and FP are widely available. Compounds of this type may be used as a direct substitute for the radioactive tracer ligands (e.g. [³H]ponasterone A) currently used in conventional ecdysone receptor based competition ligand binding assays. In this case, capture of the receptor (or of its LBD heterodimer) may be effected by one of the many ways known to those skilled in the art, such as by adsorption to glass fibre discs, or by using metal chelate-coated microtitre plate wells to capture a hexahistidine-tagged recombinant-receptor or LBD thereof, or by using microtitre plate wells coated with suitable antibodies, antibody fragments, or equivalent reagents to capture the receptor or domain. After washing the captured receptor or domain free of unbound fluorescent ligand, the amount of bound fluorescence would be determined using an appropriate instrument, such as a plate-reader capable of measuring fluorescence intensity.

More advantageously, the FP assay does not require the separation of bound from free ligand. For this and other reasons, the FP platform is highly favoured for industrial-scale high-throughput screening. A FP assay for the ecdysone receptor was not possible before the advent of the fluorescent ecdysteroid ligands provided above, nor was the mere production of a fluorescent ecdysteroid a guarantee that a useful FP assay could be devised.

Alternatively, the fluorophore can be attached to an ecdysteroid mimic, including non-steroidal ecdysone receptor agonists or antagonists such as compounds having substituted or unsubstituted dibenzoyl hydrazine chemistries. For example, a fluorescent moiety might be attached through a phenyl ring substituent or a nitrogen atom in the dibenzoyl hydrazine moiety so as to provide a fluorescent compound that interacts with an ecdysone receptor or LBD thereof.

In a third aspect the present invention provides a compound which is an ecdysteroid mimic wherein the compound comprises a non-ecdysteroid moiety that interacts with an ecdysone receptor or ligand binding domain thereof, and wherein the compound further comprises a fluorescent moiety.

Preferably the compound comprises a substituted or unsubstituted dibenzoyl hydrazine moiety that interacts with an ecdysone receptor or ligand binding domain thereof, and wherein the compound further comprises a fluorescent moiety attached through a phenyl ring substituent or a nitrogen atom in the dibenzoyl hydrazine moiety.

In a fourth aspect, the present invention provides a method for screening a candidate compound for its ability to interact with an ecdysone receptor or ligand binding domain (LBD) thereof in a competitive inhibition format, the method comprising the steps of:
(a) incubating with an ecdysone receptor or LBD thereof, a candidate compound and the compound, mimic or derivative of the invention; and
(b) measuring the level of binding of the compound, mimic or derivative of the invention to the ecdysone receptor or LBD thereof.

The FP assay of the invention includes a competitive inhibition format in which unlabelled candidate compounds (‘competitors’ or ‘inhibitors’) compete with the labelled compounds of the invention for binding to the ecdysone receptor or LBD thereof. This enables newly synthesised compounds or the compounds in existing chemical or natural-product libraries to be screened for their ability to bind to specified insect ecdysone receptors. Compounds that prove highly effective in this assay constitute lead compounds for development as insecticides against the relevant pest (and/or close relatives thereof). Preferably the competitive inhibition format is a fluorescence polarization assay.

Preferably the assay is a fluorescence polarization assay.

The invention provides scope for the development of targeted insecticides which should be attractive to agrochemical companies wishing to market a “green product” that minimises collateral damage to harmless or beneficial insects in the field. Such new insecticides should benefit from changes in the regulatory environment over recent years and could even be fast-tracked through the US registration process (USEPA PR Notice 97-3, revised PR Notice 93-9).

Accordingly, in a fifth aspect, the present invention provides an insecticidal compound identified by the assay according to the fourth aspect of the invention.

Ecdysone receptors and their functional domains are employed as components of ecdysone switches for the control of therapeutic genes in mammalian cells (Lafont & Dinan, 2003; Yang et al., 1986) and for control of transgenes more generally in agriculturally important species, both animal and plant (Lafont & Dinan, 2003; Padidam et al., 2003). The ability to screen for compound libraries against selected ecdysone receptors or the LBD thereof should aid in the discovery of safer and/or more effective ligands to act as effectors for such switches.

Accordingly, in a sixth aspect, the present invention provides an effector compound for ecdysone receptor gene switches, the compound being identified by the assay according to the fourth aspect of the invention.

The FP assay of the present invention has been tested using a commercially available fluorescence microplate reader (POLARstar Optima, BMG Labtechnologies, Germany) and shown to work with all of the recombinant ecdysone receptor LBD available to the inventors, viz. the LBD of the ecdysone receptors from Myzus persica (MpLBD), Bemisia tabaci(BtLBD), Lucilia cuprina(LcLBD), and Helicoverpa armigera (HaLBD). Indeed, a quantitative comparison of Ki values from the two assays showed that the FP screen has increased powers of discrimination over the standard radioactivity-based screen (see below).

Since each assay requires only a single microtitre plate well, and since there is no need to capture the receptor or wash away unbound tracer ligand, the process is highly amenable to automation for high-throughput screening. Moreover, FP assays are particularly amenable to miniaturization (Sportsman & Leytes, 2000) and therefore the FP assay of the present invention should be compatible with higher density multiwell formats, such as 384-well plates.

Ecdysone receptors are naturally present at very low levels in insect cells, which confounds the use of crude extracts in in vitro assays and which greatly complicates the purification of ecdysone receptors directly from insect tissue. The present invention therefore makes use of recombinantly-expressed ecdysone receptor ligand-binding regions. The inventors provide methods for purifying such recombinant proteins for use in in vitro ligand binding assays. Ligand binding preparations of this kind are particularly suitable for use in the fluorescence-based assays of the present invention.

Ecdysone receptors are present in species outside the Insecta grouping. It will be apparent to those skilled in the art that the present invention is applicable to assays/screens for ecdysone receptor ligands irrespective of the biological origin of the receptors so long as these receptors are capable of binding ecdysteroids. The ecdysone receptors may derive from members of the Insecta or other taxonomic groups within the Arthropeda or even from species within other phyla such as the Nematoda.

In order that the nature of the present invention may be more clearly understood, preferred forms thereof will now be described with reference to the following non-limiting examples.

### EXPERIMENTAL

### METHODS

Synthesis and Purification of Fluorescent Ecdysteroids

Preparation of MB4628

To a solution of 25S-inokosterone (1.3 mg, 2.5×10⁻³ mmol) [Northern Biochemical Company, Russia] in dimethylformamide (DMF) (200 μl) at 60° C. was added portionwise over 3 h fluorescein isothiocyanate isomer 1 (5.41 mg, 1.25×10⁻² mmol) [Aldrich]. The reaction was stirred at 60° C. for an additional 24 h. The DMF was removed in vacuo and the solid residue taken up in methanol (100 μl) and chromatographed using reversed-phase high pressure liquid chromatography (RP-HPLC) on a Waters chromatographic system fitted with a 150×4.6 mm Alltima C18 (5 μm) column. An aqueous solution of 65% (v/v) methanol containing 0.05% (v/v) trifluoroacetic acid was used as the mobile phase, with a flow rate of 1.0 ml/min. Absorbance peaks at 230 nm were detected using a Waters 2487 UV detector and processed using the Millennium data management system. The product, MB4628, was isolated as a solid (1.2 mg, equivalent to 55% yield) with 95% purity. NMR spectra were in accordance with the desired structure. Electrospray ionization mass spectrometry of MB4628 was performed using a single-quadrapole VG Platform with HPLC-grade methanol as solvent. The resulting spectrum showed major MS (ES) peaks at m/z 870 (M+H), 892, and 868 (M−1), consistent with expectations for the structure of MB4628 (formal mass 869.34). High resolution mass spectrometry (LSIMS) was performed using a ThermoQuest MAT 95 with a Cs gun @20 kV and a glycerol matrix. HRMS (LSIMS) (M+H) calculated for C₄₈H₅₆NO₁₂S: 870.3518. Found: 870.3519.

Other fluorescent conjugates of inokosterone were synthesised and purified by coupling methods that will be apparent to one of ordinary skill in the art. The fluorescent starting materials for some of the examples were 7-diethylaminocoumarin-3-carbonyl azide [Molecular Probes], 7-methoxycoumarin-3-carboxylic acid [Fluka Biochemica] and dansyl chloride [Aldrich]. As a result, the fluorophore moiety of MB4592 was 7-diethylaminocoumarin; for MB4603 it was 7-methoxycoumarin; and for MB4622 it was a dansyl group.

The molecular weights for inokosterone and its fluorescent conjugates were taken to be as follows: inokosterone, 480.6; MB4592, 738.9; MB4603, 697.8; MB4622, 713.9; MB4628, 869.3. Solutions of inokosterone and its fluorescent conjugates were made up by weight in ethanol (inokosterone) or methanol (fluorescent conjugates), and their molar concentrations were calculated after adjusting for the estimated purity of the compounds. The volume of each stock solution was then adjusted to give a final concentration of 1.35 mM (MB4592) or 3 mM (inokosterone, MB4603, MB4628).

Purification of the Recombinant Receptor LBDs

The method set out here has been used to purify the recombinant ligand binding portions of ecdysone receptors from several species of commercially important insect pests. The method provides active material in sufficient quantities for use in in vitro ligand-binding assays, including the fluorescence-based assays of the present invention.

The LBDs of the EcR and USP subunits from each insect species were co-expressed in cultured insect cells using a recombinant baculovirus. To facilitate their detection and purification, each recombinant EcR LBD had been engineered to contain a hexahistidine (His₆) affinity tag at its N-terminus, and each recombinant USP LBD had been engineered to contain a FLAG affinity tag at its N-terminus. The hexahistidine affinity tag allowed the recombinant EcR/USP LBD heterodimer to be purified on a preparative scale by IMAC chromatography. To obtain recombinant receptor LBD suitable for use in in vitro ligand-binding assays, the extraction and immobilised metal-ion affinity chromatography (IMAC) purification was done in the absence of added ecdysteroids or other EcR ligands. The non-denaturing detergent CHAPS was sometimes included up to (but not during or after) the IMAC wash step in an attempt to minimise the amount of an unwanted protein (approx. 75 kDa) that tended to co-purify with the recombinant LBD, irrespective of what species the recombinant receptor LBD came from. Further purification of the recombinant receptor LBDs was possible, for example by subjecting the IMAC-purified material to ion exchange chromatography (e.g., Pharmacia Mono-Q) or gel filtration (e.g., Pharmacia Superdex-200), but was not considered necessary for the present invention.

The construction of baculoviruses for expression of functional EcR/USP LBDs from the ecdysone receptors of the sheep blowfly, Lucilia cuprina, peach aphid, Myzus persicae, and silverleaf whitefly, Bemisia tabaci are described in the two patent families directed towards a range of EcR and USP ecdysone receptor subunits (PCT/AU99/00033 and PCT/AU00/00799) and the Australian Provisional Application number 2003902621. The baculovirus for expression of the EcR/USP LBD heterodimer of the cotton bollworm, Helicoverpa armigera, was constructed by similar methods from cDNAs encoding HaEcR and HaUSP closed in the inventors laboratory.

Pilot-scale expression of recombinant EcR/USP LBD heterodimer was achieved by infection of suspension cultures of Sf9, Sf21 and or Hi-5 insect cells in spinner flasks or Schott bottles on a shaker platform maintained at 27° C. Insect cells infected with the virus engineered to express the EcR/USP LBD heterodimer were shown by gel electrophoresis to contain the expressed polypeptides corresponding to the two tagged domains. In ligand binding assays (adapted from Koelle et al., 1991) the recombinant cell lysates had a greatly enhanced ability to bind the radiolabelled-ecdysteroid, [³H]ponasterone A, compared to control cell lysates. These results indicated that the recombinant virus was expressing functional domains that were able to heterodimerise and form a recombinant receptor LBD heterodimer that bound ecdysteroids with high affinity.

Large-scale recombinant protein production was carried out by infecting insect cells in a 6L stirred bioreactor. Typically, baculovirus-infected Hi-5 cells were grown in a Celligen fermentor (New Brunswick Scientific) under controlled conditions (27° C., 35 r.p.m.). The identity, integrity and purity of the recombinant domains was monitored during downstream processing by SDS-polyacrylamide gel electrophoresis (PAGE), using Coomassie stain to visualise total protein or immunoblotting (with anti-tag antibodies) to visualise just the domains. By way of example, we will now describe the extraction and purification procedure for MpLBD, the recombinant heterodimeric EcR/USP LBD from the ecdysone receptor of M. persicae.

A recombinant baculovirus that had been engineered to co-express the EcR and USP subunits of the MpLBD heterodimer was amplified and used to infect a 4.5 litre culture of Hi-5 insect cells in the Braun Bioreactor with a multiplicity of infection of approximately 5. Harvested at 49 h post-infection, this culture yielded 37 g wet weight of recombinant insect cells, which were snap-frozen in liquid nitrogen and stored at −70° C. The entire batch of cells was later thawed and suspended in 170 ml EcR40 buffer [25 mM Hepes, 40 mM KCl, 10% glycerol, 1 mM sodium EDTA, 3 mM sodium azide] containing 2.1 μM leupeptin, 2.0 μM pepstatin, 0.95 mM phenylmethanesulphonyl fluoride, 19.5 mM Na₂S₂O₅, 1.9 mM CHAPS, 9.6 mM 2-mercaptoethanol, pH 7.0, 4° C.) and sonicated to break open the cells (4 batches of equal volume, each treated with 14×5 sec pulses, with 25 sec cooling in salted ice between each pulse, on a MSE Type 1174.MK2 sonicator fitted with a 19 mm diameter probe). The sonicates were recombined (215 ml total volume) and the ionic strength was then raised by addition of 20.8 ml 4 M KCl. This sample was ultracentrifuged to pellet cellular debris (Beckman 60Ti rotor in Beckman L8-80M Ultracentrifuge: 100 000 g, 1 h, 4° C.). The supernatant was dialysed (Spectrum Spectra/Por 1 tubing, 40 cm long×5 cm diameter) for 3 h at 4° C. against 1100 ml EcR40 buffer containing 10 mM 2-mercaptoethanol to lower the ionic strength. The dialysate (which had become cloudy) was clarified by centrifugation (Beckman JA14 motor in Beckman J2-21 centrifuge, 12 000 rpm, 30 min; 4° C.). It was then snap-frozen in liquid nitrogen and stored at −70° C. To resume the purification, the sample was thawed rapidly (by shaking in a 37° C. water bath) and dialysed (Spectrum Spectra/Por 1 tubing, 40 cm long×5 cm diameter) twice for 3 h at 4° C. against 1100 ml phosphate buffer (50 mM sodium phosphate, 10% glycerol, 0.3 M NaCl, 2.4 mM CHAPS, 10 mM mercaptoethanol, 3 mM sodium azide, pH 7.4). The dialysate (200 ml total) was then snap-frozen in liquid nitrogen and stored at −70° C.

The frozen dialysate was thawed rapidly (by shaking in a 37° C. water bath) and re-clarified (Beckman JA14 rotor in Beckman J2-21 centrifuge, 12 000 rpm, 20 min, 4° C.). To the clarified protein sample was added 2 ml 2M imidazole, pH 7.4, containing 3 mM sodium azide. A 6 ml portion of a 50% slurry of Ni-NTA agarose beads (Qiagen, Cat. No. 30210) was washed twice with 20 ml phosphate buffer (50 mM sodium phosphate, 10% glycerol, 0.3 M NaCl, 10 mM 2-mercaptoethanol, 3 mM sodium azide, pH 7.4). The washed beads were combined with the protein sample and the suspension was rotated slowly (RotoTorque: 10 rpm, 3 h, 4° C.). The beads were then pelleted by centrifugation (Beckman JA14 rotor in Beckman J2-21 centrifuge, 10 000 rpm, 20 min, 4° C.). The supernatant was removed carefully, after which the beads were transferred to a mini-column (a 10 ml syringe body clamped upright, with a disc of Whatman filter-paper serving as a frit at the base) at 4° C. Unbound proteins were removed by washing the column of beads with 70 ml phosphate buffer (50 mM sodium phosphate, 10% glycerol, 0.3M NaCl, 10 mM 2-mercaptoethanol, 20 mM imidazole, 3 mM sodium azide, pII7.4) at 4° C. Specifically-bound proteins were eluted with a buffer containing a high imidazole concentration (50 mM sodium phosphate, 10% glycerol, 0.3 M NaCl, 10 mM 2-mercaptoethanol, 250 mM imidazole, 3 mM sodium azide, pH 7.4). To maximise recovery, the elution buffer was applied to the column as 2×23 ml aliquots with a 20 min interval between each application. The eluates were combined and the pool was divided into aliquots, snap-frozen in liquid nitrogen, and stored at −70° C. A portion was assayed for protein content using the Pierce Coomassie Plus assay, calibrated using bovine serum albumin. Protein concentrations determined in this way were known to be within 6% of those determined by quantitative amino acid analysis (data not shown). Molar concentrations were calculated using the expected molecular mass for each heterodimeric LBD (i.e. the sum of its two conceptually translated LBD polypeptides), as follows: LcLBD, 81.5 kDa; Mp, 68.2 kDa; BtLBD, 65.8 kDa; HaLBD, 74.2 kDa.

Similar procedures were used to prepare LcLBD, BtLBD, and HaLBD, although in some cases CHAPS was omitted from the procedure. Comparative tests (not shown) confirmed that transient exposure of the unliganded recombinant receptor LBD heterodimers to CHAPS during their extraction and IMAC capture had no effect on the ligand-binding activity of the final (CHAPS-free) preparation. In contrast, and as discussed in greater detail below, the presence of CHAPS in ligand binding assays often improved the proteins apparent ligand-binding capacity.

For each recombinant receptor LBD heterodimer, equilibrium binding experiments were performed in which different concentrations of [³H]ponasterone A (typically 0.1-4.0 nM bindable radioligand) were incubated with a low fixed concentration of the LBD heterodimer, and receptor-radioligand binding was determined essentially as described below (viz. the manual procedure for MpLBD in ‘METHODS—Testing fluorescent and reference ecdysteroids as ligands using a radioligand-based assay’, performed in a total volume of 154 μl but with no competitor ligand present). Since the condition [[3H]ponA]_{bound}<[[3H]ponA]ₜₒₜₐₗ/10 was observed, ligand depletion was avoided and plots of bound vs. free [3H]ponasterone A concentrations were able to be fitted directly to the Langmuir isotherm (Hulme & Birdsall, 1992)
[[3H]ponA]_{bound}=[[3H]ponA]_{free}−[LBD]ₜₒₜ/([K_{d}+[[3H]ponA]_{free})
where [[3H]ponA]ₜₒₜₐₗ is the total concentration of bindable [³H]ponasterone A in the assay, [[³H]ponA]_{free} is the concentration of free bindable [³H]ponasterone A in the assay, [[3H]ponA]_{bound} is the concentration of bound [³H]ponasterone A, [LBD]ₜₒₜ is the total concentration of functional recombinant receptor LBD heterodimer, and K_{d} is the dissociation constant for [³H]ponasterone A with that receptor under the conditions of the assay. Data fitting using a computer algorithm (KalcidaGraph v3.09, Synergy Software) allowed us to obtain Kd values for each receptor-radioligand complex and to ascertain [LBD]ₜₒₜ. Comparison of the [LBD]ₜₒₜ value with the protein concentration of each purified sample indicated the proportion of the recombinant receptor LBD heterodimer molecules that was functional.
Testing Fluorescent and Reference Ecdysteroids As Ligands Using a Radioligand-based Assay

The ability of inokosterone and fluorescent conjugates thereof to bind to recombinant M. persicae ecdysone receptor was assessed using the conventional radioligand binding assay in a competetive inhibition format (adapted from Koelle et al., 1991). Thus (1) increasing amounts of inokosterone or fluorescent conjugate thereof were added to assay mixtures containing fixed concentrations of MpLBD and [³H]ponasterone A; (2) after equilibration, the MpLBD (including the MpLBD-ligand complexes) was captured by adsorption onto a glass-fibre filter and washed free of unbound [³H]ponasterone A; and (3) the amount of radioligand bound by the receptor was determined by scintillation counting the filter.

These procedures were performed manually, as follows. Assays were performed in EcR40 buffer [25 mM Hepes, 40 mM KCl, 10% glycerol, 1 mM sodium EDTA, 3 mM sodium azide] containing 0.5 mg/ml bovine serum albumin (BSA). An extract containing recombinant M. persicae edcdysone receptor, MpLBD, was prepared as described above and diluted in EcR40 buffer containing 0.5 mg/ml BSA to a concentration that generated filter counts around 10 000 cpm when the assay was done in the absence of competing ligands. The [³H]ponasterone A ([24,25,26,27-³H(N)]ponasterone A, NEN Life Sciences, Cat. No. NET-1070) was present at a final concentration of 2.0 nM, after adjustment for the proportion of radioactivity (typically 30%) that remained unbindable even at extremely high receptor concentrations. Assays were set up to contain different final concentrations of fluorescent ecdysteroid by including the appropriate volumes of the relevant stock solutions. The final concentration of ethanol or methanol in the incubation mixture did not exceed 0.8% (v/v), a level known not to significantly affect the extent of radioligand binding (data not shown). Each incubation was performed in a final volume of 166 μl at room temperature (22° C.) for 90 min, whereupon it was held on ice until the filter adsorption/wash steps could be performed. Three 140 μl aliquots from each completed incubation were applied to glass microfibre filters (GF/C 24 mm diameter, Whatman, Cat. No. 1822024); i.e., every incubation generated three filters, each of which had been wet using 140 μl of the incubation mixture. After exposure to the liquid for 30 sec at room temperature, the wet filter was transferred to a vacuum sinter apparatus (Pyrex Filter Holder, Millipore Corp., Cat. No. XX1002500) and washed rapidly. Washing was done using 2×5 ml of ice-cold EcR40 buffer, intersperced with 3×0.5 ml buffer applied around the circumference of the filter to ensure that its edges were thoroughly rinsed. The damp filter was then transferred to a scintillation vial. When all the filters had been thus processed, 7 ml Packard InstaGel Plus scintillant was added to each scintillation vial. The sealed vials were vortexed and then incubated at room temperature for at least 2 h, during which time the filters became transparent. The vials were then scintillation counted (1 min per filter, tritium program, Packard TriCarb 2100TR scintillation counter). Datapoints were reported as the mean cpm value ±SEM for the three replicate filters arising from each incubation. To enable a comparison of the different inokosterone derivatives, the cpm data from individual titrations were converted to % activity values, where 100% activity was equivalent to the cpm value obtained in the absence of non-radioactive competitor compound, and 0% activity was equivalent to the background cpm value obtained in the absence of MpLBD.

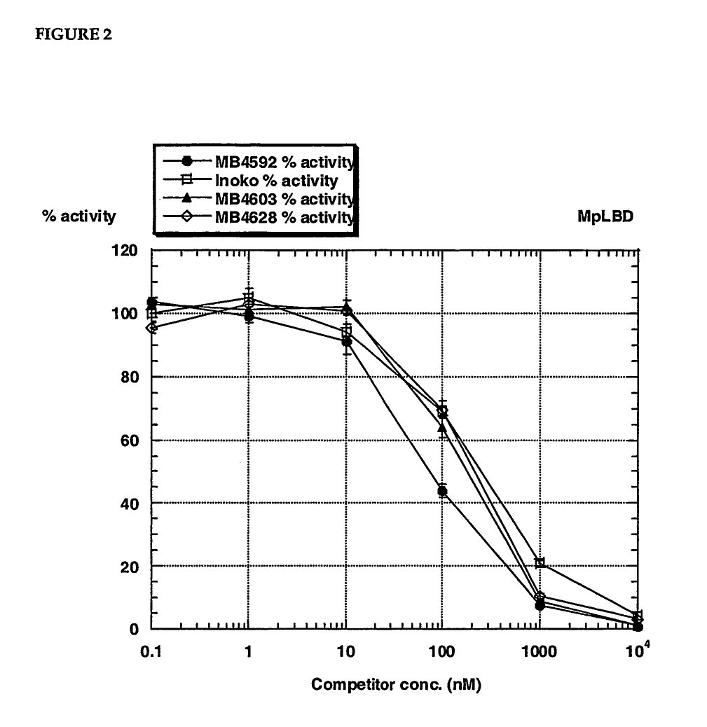

Smooth curves were drawn for the radioligand assay competitive inhibition curves using a Flexicurve; these represent a more realistic fit to the data than the linear point-to-point plots shown (for convenience of printing) in FIG. 2. IC₅₀ values were calculated from the smooth titration curves using the midpoints between the 100% and 0% activity values. The inhibitor concentration at this midpoint was deemed to be the IC₅₀ value for the inhibitor. IC₅₀ values were converted to Kᵢ values using the Cheng-Prusoff equation (Cheng & Prusoff, 1973), as follows:
Kᵢ=IC₅₀/(1+([[³H]ponA]ₜₒₜ/K_{d}))
where [[³H]ponA]ₜₒₜ is the total concentration of bindable [³H]ponasterone A in the assay, and K_{d} is the dissociation constant for [³H]ponasterone A with the receptor under the conditions of the assay. The [[³H]ponA]ₜₒₜ value was 2.0 nM, as mentioned above, while for MpLBD a value of 0.7 nM was used for K_{d} (see ‘RESULTS—Purification of the recombinant receptor LBD's).

Subsequently, a miniaturized and automated version of the radioligand-based competition assay was used with MpLBD and BtLBD to obtain Kᵢ values for the non-radioactive, non-fluorescent ecdysteroids that would later be used to validate the competitive inhibition format of the FP assay. While the concept of the assay was unchanged from that described above, some aspects of its execution were different. In this case, the assays were performed in 96-well plates (V-bottomed polypropylene, Greiner Bio-One, Cat. No. 651201), the bindable [³H]ponasterone A concentration was 1.3 nM, the MpLBD or BtLBD concentration was designed to give around 2000 cpm per filter in the absence of competing ligands, the total volume of each assay was 30 μl, and the incubation mixtures were prepared by a Beckman Biomek 2000 robotic workstation. The mixtures were again allowed to reach equilibrium, but this time for 4 h at room temperature. The MpLBD was then captured on a 96-filter array (Unifilter-96 GF/C, Packard, Cat. No. 6005174) and rinsed using the wash tool and vacuum block of the Biomek 2000. The back of each filter-plate was then manually sealed using a sheet of Packard BackSeal, and 25 μl Packard MicroScint-20 scintillation fluid was dispensed into each well. The top of each plate was sealed with Packard TopSeal-A and the filters were allowed to solvate overnight. The amount of bound [³H]ponasterone A was then measured using a Packard TopCount scintillation counter (1 min per filter, tritium/Microscint program). IC₅₀ and Kᵢ data were derived as before, except that 1.3 nM was used for [³H]ponasterone A concentration in the Cheng-Prusoff equation. Since the amount of bound radioactivity was at most 28% of the total present, no additional corrections for ligand depletion were applied.

Monitoring Fluorescent Ecdysteroid Binding By FP

The standard FP assay buffer was 50 mM sodium phosphate, 100 mM NaCl, pH 7.4, containing 0.5 mg/ml bovine serum albumin. When required, preparative pipetting steps (such as dilution series) were typically done in conventional V-bottomed 96-well microplates (e.g. V-bottomed polypropylene, Greiner Bio-One, Cat. No. 651201). The assays themselves were set up in opaque black flat-bottomed 96-well plates designed for fluorescence measurements (e.g. Nunc, Cat. No. 237108). The final volume of all assays was 250 μl. The FP plate-reader was a POLARstar Optima (Cat. No. 413-201, BMG Labtechnologies, Offenburg, Germany) with fluorescence polarization optics (installed according to the manufacturer's instructions) and operated by FLUOstar Optima software in Plate Mode (Polarization). Excitation was done at 485 nm (filter 485) and emission was detected at 520 nm (filter 520p). The standard instrument setup involved a 3 sec/3 mm shake for the plate prior to commencing reading; readings used 200 flashes/assay. Gain values were typically around 3000 for each channel, with a K-factor close or equal to 1.0.

A 250 μl sample of 30 nM fluorescein in 50 mM sodium phosphate, 100 mM NaCl, pH 7.4, was known to have a polarization value of 35 mP and was used to calibrate the FP plate-reader. The instrument indicated that a 250 μl sample of 36 nM MB4628 in the same buffer had a polarization value close to 100 mP. Thereafter, at the commencement of each experimental session, the FP plate-reader was adjusted to give a reading of 100 mP for a 250 μl sample of 36 nM MB4628 in standard FP assay buffer.

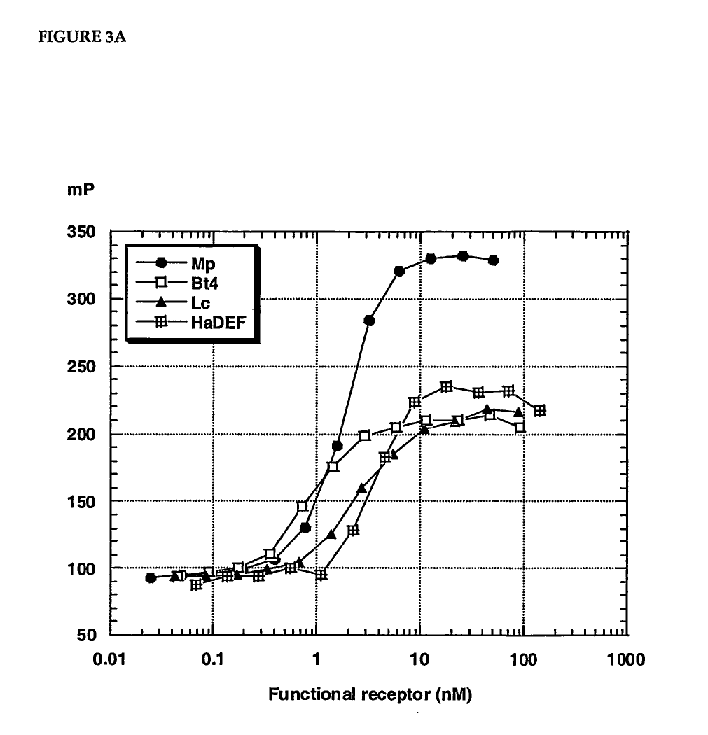

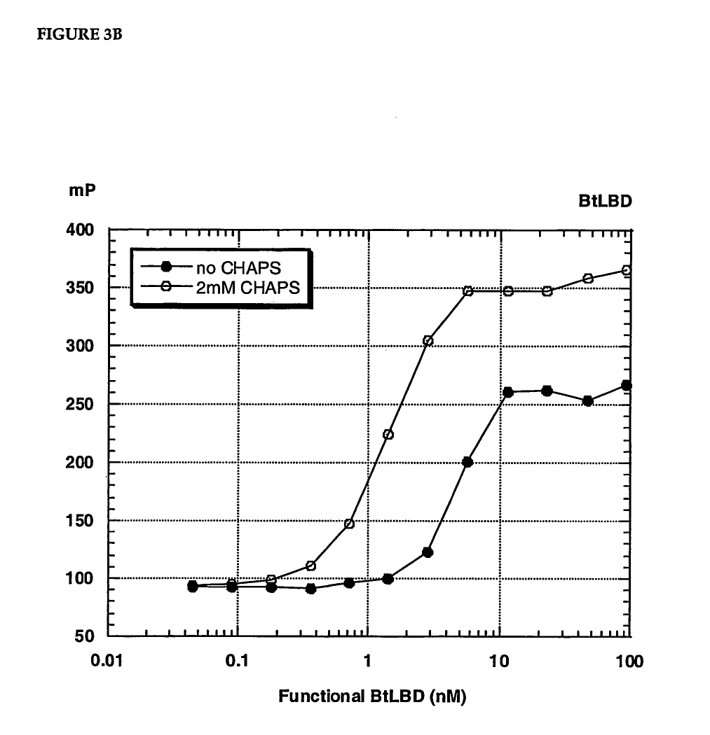

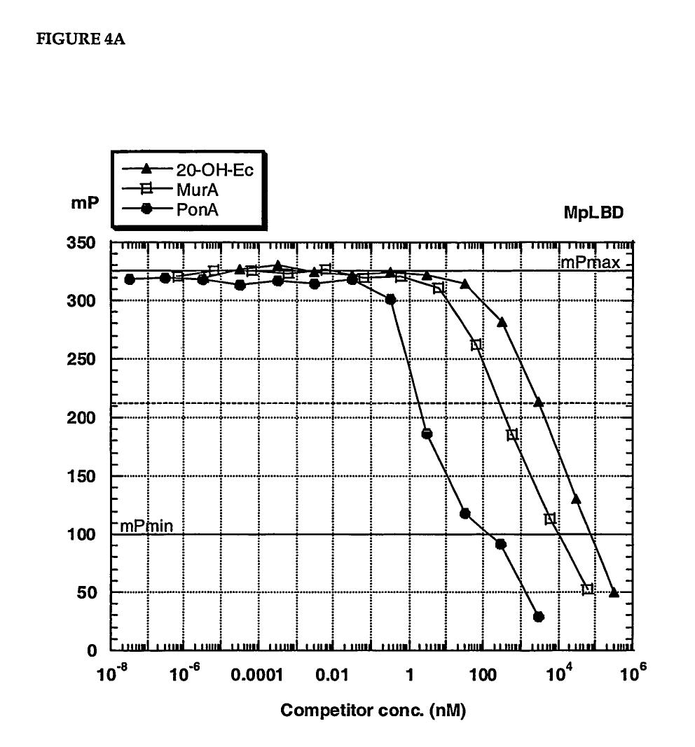

Receptor LBD titrations involved mixing a small volume (e.g. 2.5 μl) of diluted receptor LBD heterodimer stock with a larger volume (e.g. 247.5 μl) of standard FP assay buffer containing 36 nM MB4628. Where indicated, CHAPS was present at a final concentration of 2 mM. Receptor dilutions were typically arranged to cover a wide concentration range, e.g. 0.005-50 nM functional receptor. The assay mixtures were allowed to reach equilibrium, typically by incubating overnight at 4° C. and then equilibrating at room temperature (20° C.) for 2-4 h before reading the mP values. When plotting the data, smooth curves were drawn for the FP titrations using a Flexicurve; these represent a more realistic fit to the data than the linear point-to-point plots shown (for convenience of printing) in FIG. 3.

Tests showed that higher concentrations of tracer ligand did not significantly improve the dynamic range of the assay, but did increase the amounts of both receptor and tracer required to perform the assay (data not shown). Other tests showed that the small amounts of additives carried over from the receptor stocks (in IMAC column elution buffer) into the FP assays did not have a significant effect on the mP value (data not shown).

Compound libraries typically comprise arrays of stock solutions in an organic solvent, such as dimethylsulphoxide (DMSO) or ethanol (EtOH). Before examining the effects of competitive inhibitors on the binding of MB4628 by recombinant ecdysone receptors, it was necessary to determine the effects of such solvents on the FP assay. Accordingly, FP assays containing either no receptor or 1.5 nM functional MpLBD were tested with EtOH up to 1% (v/v) and DMSO up to 6% (v/v).

Previous experiments (not shown) with the radioligand-based assay had indicated that the presence of 2 mM 3-[(3-cholamidopropyl) dimethylammonio]-1-propanesulphonate (CHAPS) in the assay could increase by 2- to 4-fold the [³H]ponasterone A-binding capacities of LcLBD and MpLBD, apparently without altering the K_{d} values for this ligand. It was therefore considered appropriate to test whether the inclusion of 2 mM CHAPS in the FP assay might improve its performance, for example by increasing its dynamic range.

Screening Compound Libraries For Ecdysone Receptor Ligands By FP

Before reading assay wells that did not contain CHAPS, the FP plate-reader was adjusted to give a MP reading of 100 for a well containing 250 μl 36 nM MB4628 in standard FP assay buffer. Before reading assay wells containing 2 mM CHAPS, the FP plate-reader was adjusted to give a mP reading of 100 for a well containing 250 μl 36 nM MB4628 plus 2 mM CHAPS in standard FP assay buffer.

For competitive inhibition assays, each well contained standard FP assay buffer containing 36 nM MB4628 and either 5 nM functional MpLBD or 6 nM functional BtLBD. The BtLBD assays, but not the MpLBD ones, were routinely done in the presence of 2 mM CHAPS. The final volume of all assays were 250 μl. Under the conditions described here, when no competing ligand is present the observed mP value is close to the maximum possible value for the assay. To provide similar conditions for LcLBD in the absence of CHAPS, we suggest 250 μl standard FP assay buffer containing 36 nM MB4628 and 20 nM functional LcLBD. Likewise, for HaLBD we suggest 250 μl standard FP assay buffer containing 36 nM MB4628 and 8 nM functional HaLBD. Note that the inclusion of CHAPS is likely to improve the performance of LcLBD and HaLBD competitive inhibition assays (see RESULTS).

Smooth curves were drawn for the FP competitive inhibition curves using a Flexicurve; these represent a more realistic fit to the data than the linear point-to-point plots shown (for convenience of printing) in FIG. 4. IC₅₀ values were calculated from the smooth titration curves using the midpoint between the actual maximum mP value (mPₘₐₓ) and the theoretical minimum mP value (mPₘᵢₙ), even where the actual plot deviated from theoretical minimum mP value (mPₘᵢₙ), even where the actual plot deviated from theoretical expectations at high inhibitor concentrations by having mP values below 100 mP. The inhibitor concentration at this midpoint was deemed to be the IC₅₀ value for the inhibitor. IC₅₀ values were converted to Kᵢ values using the Cheng-Prusoff equation (Cheng & Prusoff, 1973), as follows:
Kᵢ=IC₅₀/(1+([MB4628]ₜₒₜ/K_{d}))
where [MB-4628]ₜₒₜ is the total concentration of MB4628 in the assay, and K_{d} is the dissociation constant for MB4628 with the relevant receptor under the conditions of the assay. The K_{d} values for use in this equation were calculated from the basic FP titration curves (FIG. 3) using the knowledge that the receptor concentration at the titration midpoint is the value at which the receptor is half-saturated with MB4628. The K_{d} is the free MB4628 concentration at this point, and must therefore be:
K_{d}=[MB4628]ₜₒₜ−([receptor]_{midpoint})/2

The K_{d} value used here was 35.3 nM for both MpLBD (without CHAPS) and BtLBD (with 2 mM CHAPS). Note that the K_{d} value for MB4628 derived from its (CHAPS-free) FP titration curve with MpLBD can be compared directly with the Kᵢ value determined by the same ligand's ability to compete with [³H]ponasterone A for (CHAPS-free)binding to the same receptor (see ‘RESULTS—Testing fluorescent and reference ecdysteroids as ligands using a radioligand-based assay’). The two values, 35.3 nM and 40.0 nM respectively, are in close agreement. This is consistent with theoretical expectations, since both constants describe the dissociation of the MpLBD-MB4628 complex.

The effect of omitting CHAPS from BtLBD competitive inhibition assays was tested directly. Thus, in a BtLBD experiment that departed from standard procedure, 10 nM functional BtLBD was incubated with 36 nM MB4628 and increasing concentrations of non-fluorescent ecdysteroids, but without CHAPS. The K_{d} values for MB4628 under these conditions, 34.7 nM, was used when calculating Kᵢ values employing the Cheng-Prusoff equation.

### RESULTS

Purification of the Recombinant Receptor LBDs

Successful 4-6 L baculovirus infected Hi-5 insect cell cultures yielded 70-100 g wet cells, which typically contained about 0.3 mg recombinant LBD protein per gram cells. The IMAC purification of MpLBD described in detail in the METHODS section yielded 17 mg of purified protein from 37 g wet cells.

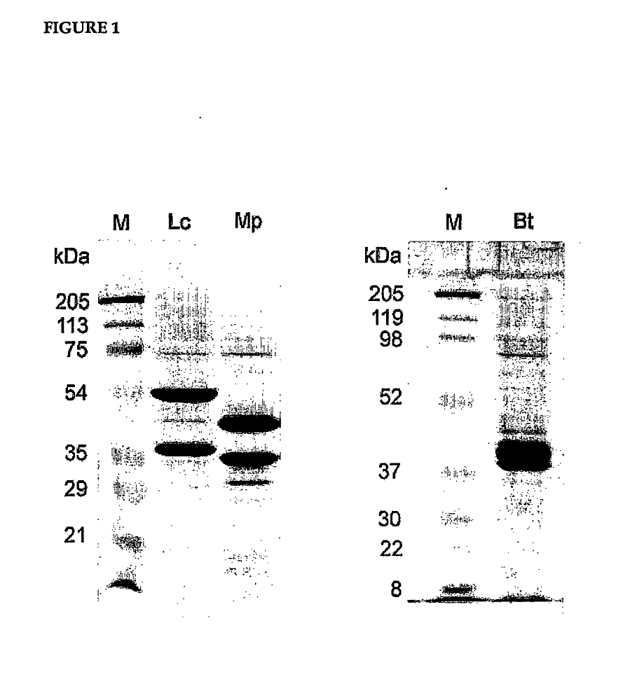

An analysis of the IMAC-purified receptor LBD heterodimers by reducing SDS-PAGE suggested that the preparations were over 90% pure, and confirmed that the recombinant EcR and USP polypeptides were present in approximately equimolar amounts (FIG. 1). The apparent molecular mass of each recombinant subunit was close to that predicted for the polypeptide encoded by the relevant sub-gene, although all of the recombinant species migrated slightly more slowly than expected (additional data not shown).

For each recombinant receptor LBD heterodimer preparation, equilibrium binding experiments with [³H]ponasterone A (not shown) gave estimates of K_{d} as follows: LcLBD, K_{d}=1.0±0.10 nM; MpLBD, K_{d}=0.72±0.09 nM; BtLBD, K_{d}=1.21±0.17 nM; HaLBD, K₃=2.53±0.12 nM. A similar binding study done using L. cuprina embryo extracts (not shown) gave K_{d}=0.92±0.10 nM, thereby confirming that the ligand-binding function of the recombinant LBD was, within experimental error identical to that for the non-recombinant (full-length) ecdysone receptor.

The same equilibrium binding experiments indicated the properties of functional receptor LBD heterodimer molecules in each preparation to be as follows: LcLBD, 21%; MpLBD, 16%; BtLBD, 8.4%. The value for HaLBD was not determined directly, but by indirect methods it was assigned a provisional value of 15%. The relatively low activity values agree with published observations for the full-length Drosophila melanogaster receptor, for which Sage et al. (1986) remark that ‘. . . the binding activity of unloaded receptor is inherently labile when subjected to standard protein purification techniques. Therefore, the ecdysteroid receptor needs to remain loaded with hormone during most manipulations’. While we agree that a much higher proportion of functional receptor protein can be obtained by conducting the purification in the presence of an ecdysteroid ligand (data not shown), the resulting receptor-ligand complex is not suitable for use in ligand-binding assays. Moreover, bound ecdysteroid ligands are slow to dissociate from the recombinant receptor LBDs. For example, attempts to remove bound [³H]ponasterone A from LcLBD by dialysis at room temperature showed that the half-life of the radioligand-receptor complex was about 10 h (data not shown). We therefore routinely purified the recombinant receptor LBD heterodimers in the absence of any ligands, and corrected the concentration values of the purified LBD heterodimer preparations to account for the proportion of non-functional heterodimer that was present. Concentrations corrected in this way are expressed in terms of “nM functional receptor”.

Testing Fluorescent and Reference Ecdysteroids As Ligands Using a Radioligand-based Assay

Inokosterone and its fluorescent conjugates were tested for the ability to compete with [³H]ponasterone A for binding to MpLBD, the recombinant ecdysone receptor from M. persicae. Under these circumstances, titrations of inokosterone and its fluorescent derivatives conjugated via C-26, MB4603, MB4592, and MB4628, all gave similar sigmoid curves (FIG. 2). The midpoints of the curves indicate Kᵢ values of 65 nM for inokosterone, 40 nM for MB4603, 20 nM for MB4592, and 40 nM for MB4628. The dansyl derivative of inokosterone, MB4622, was also a highly effective ligand for MpLBD (data not presented). On the other hand, a derivative with a dansyl moiety conjugated via C-3 of the steroid A-ring, MB4588, did not compete with [³H]ponasterone for binding to MpLBD (data not shown).

Similar assays (not shown) using [³H]ponasterone A and either MpLBD or BtLBD were used to obtain Kᵢ values for the non-radioactive, non-fluorescent ecdysteroids that would later be used to validate the competitive inhibition format of the FP assay. For MpLBD, the Kᵢ values for ponasterone A, muristerone A and 20-hydroxyecdysone were determined to be 0.28, 30 & 100 nM, respectively. For BtLBD, the Kᵢ values for ponasterone A, muristerone A and 20-hydroxyecdysone were determined to be 4.8, 5.3 & 240 nM, respectively. Note that the Kᵢ value for ponasterone A with MpLBD (˜0.3 nM) is comparable to the K_{d} value reported above for [³H]ponasterone A with MpLBD (˜0.7 nM; see ‘RESULTS—Purification of the recombinant receptor LBDs). Likewise, the Kᵢ value for ponasterone A with BtLBD (4.8 nM) is not very different from the K_{d} value reported above for [³H]ponasterone A with BtLBD (1.2 nM; see ‘RESULTS—Purification of the recombinant receptor LBDs). The agreement between the Kᵢ and K_{d} values for each receptor is consistent with theoretical expectations, since in each case the two constants describe the dissociation of the same receptor-ligand complex.

FIG. 2 shows that all three of the fluorescent conjugates of inokosterone, MB4603, MB4592, and MB4628, were unimpaired in their ability to bind to the recombinant ecdysone receptor. The data suggest a model in which the fluorescent chromophore does not exert significant steric or electronic influence on the binding of the ecydsteroid moiety to the receptor. Of the compounds tested, the special properties of the fluorescein conjugate (MB4628) made it the ligand of choice for developing a fluorescence-based assay. We therefore developed a novel assay that focused on MB4628 and exploited FP to monitor the binding of this ligand to recombinant ecdysone receptor LBD heterodimers.

Monitoring Fluorescent Ecdysteroid Binding by FP

The mP value for free MB4628 in standard FP assay buffer was 100, so for all assays the minimum value was 100 mP (mPₘᵢₙ=100 mP). Titrations of 36 nM MB4628 with MpLBD gave sigmoid curves where the maximum mP value was 335 mP (mPₘₐₓ=335 mP) and the curve midpoints corresponded to 1.1-1.8 nM functional receptor (FIG. 3A). In terms of FP assays, a dynamic range of 235 mP is considered to be excellent. A titration of 36 nM MB4628 with LcLBD gave a sigmoid curve with mPₘₐₓ=220 mP (i.e. about half the dynamic range of MpLBD) and a midpoint of 2.5 nM functional receptor (FIG. 3A). Similar titrations with BtLBD gave sigmoid curves with mPₘₐₓ=210-260 mP and a midpoint of 1 to 5 nM functional receptor (FIG. 3A). Similar titrations with HaLBD gave a sigmoid curve with mPₘₐₓ=230 mP (similar to LcLBD & BtLBD, and substantially lower than for MpLBD) and a midpoint of 3.5 nM functional receptor.

The FP assay showed a 6% depression of mP value at final concentrations of 1% (v/v) EtOH, which was not considered significant. Since the competitive inhibition assays reported here involved final concentrations at or below 1% (v/v) EtOH, no correction for solvent effects was applied. In contrast, the FP assay was significantly affected by DMSO, showing 18% depression of mP value at final concentrations of 1% DMSO (v/v) and 41% depression of mP value at 6% DMSO (v/v). Since this effect occurs irrespective of whether or not any ecdysone receptor is present, DMSO should have little impact on the intrinsic dynamic range of the FP assay. However, in addition to this effect, some of the recombinant receptor LBDs are intrinsically sensitive to DMSO. Thus, radioligand-based assays (not shown) reveal that, while ligand binding to LcLBD or MpLBD is largely unaffected by DMSO, binding to BtLBD is ˜50% inhibited by a final concentration of 6% (v/v) DMSO and HaLBD is ˜30% inhibited by a final concentration of 1% (v/v) DMSO. While this may limit the ability to screen DMSO-based compounds libraries for modest or poor ligands with the more DMSO-sensitive receptors, it is worth pointing out that conventional radioactivity-based assays suffer from exactly the same limitation.

Performing a BtLBD titration in the presence of 2 mM CHAPS did not increase the absolute value of mPₘₐₓ but did decrease the mPₘᵢₙ value to below zero. Therefore, the FP plate-reader had to be re-standardised to 100 mP using a well containing 250 μl of standard FP assay buffer containing 36 nM MB4628 and 2 mM CHAPS (without receptor) before attempting to read CHAPS-containing assay wells. The dynamic range of a BtLBD titration done in the presence of 2 mM CHAPS (approximately 260 mP) was now excellent, and similar to that of a (CHAPS-free) MpLBD titration (FIG. 3B). The presence of CHAPS also significantly lowered the BtLBD titration midpoint: in the current experiment, the midpoint shifted from a CHAPS-free value of 4.5 nM to a new value of 1.5 nM functional BtLBD when 2 mM CHAPS was present. This effect may be helpful in minimising receptor consumption during screening. In contrast, 2 mM CHAPS caused only a slight increase in the dynamic range of a MpLBD assay and caused a 1.5-fold increase in the titration midpoint.

Screening Compound Libraries For Ecdysone Receptor Ligands By FP

For the M. persicae receptor, competitive inhibition was detected by incubating 5 nM functional MpLBD with 36 nM MB4628 in the presence of increasing concentrations of non-fluorescent ecdysteroids (FIG. 4A). High concentrations of unlabelled ecdysteroids depressed the observed mP values below that for free MB4628, suggesting some interference with MB4628 fluorescence, a phenomenon which was not observed in the absence of receptor (data not shown). Even when the lower boundary was set to the theoretical value for free MB4628, the useable dynamic range of the MpLBD assay (225 mP) was excellent. The IC₅₀ values extracted from the MpLBD curves for inhibition by ponasterone A, muristerone A and 20-hydroxyecdysone converted into Kᵢ values of 1, 149 & 1490 nM, respectively. As described above (see ‘RESULTS—Testing fluorescent and reference ecdysteroids as ligands using a radioligand-based assay’), the corresponding Kᵢ values from radioligand-based MpLBD assays were 0.28, 30 & 100 nM. Thus, despite the differences in absolute values, the FP assay ranked the competitors correctly in terms of potency and displayed increased powers of discrimination over the radioligand-based assay. The FP-derived Kᵢ value for each unlabelled ecdysteroid was plotted against the corresponding Kᵢ value from the radioligand-based assay (FIG. 5).

For the B. tabaci receptor, competitive inhibition was detected by incubating 6 nM functional BtLBD with 36 nM MB4628 in the presence of 2 mM CHAPS and increasing concentrations of non-fluorescent ecdysteroids (FIG. 4B). In these titrations, high concentrations of unlabelled ecdysteroids did not depress the mP values below that for free MB4628. The useable dynamic range of the BtLBD assay (225 mP) was excellent. The IC₅₀ values extracted from the BtLBD curves for inhibition by ponasterone A, muristerone A and 20-hydroxyecdysone converted into Kᵢ values of 7.4, 27, & 994 nM, respectively. As described above (see “RESULTS—Testing fluorescent and reference ecdysteroids as ligands using a radioligand-based assay”), the corresponding Kᵢ values from radioligand-based BtLBD assays were 4.8, 5.3 & 240 nM. Thus the FP assay again ranked the competitors correctly in terms of potency, and displayed increased powers of discrimination over the radioligand-based assay. The FP-derived Kᵢ value for each unlabelled ecdysteroid was plotted against the corresponding Kᵢ value from the radioligand-based assay (FIG. 5).

The detrimental effect of omitting CHAPS from BtLBD competitive inhibition FP assays was demonstrated in an experiment that departed from the standard procedure for this receptor. In this case (data not shown), high concentrations of unlabelled ecdysteroids depressed the mP value below that for free MB4628, just as they had done in the CHAPS-free MpLBD assays. The useable dynamic range of the CHAPS-free BtLBD assay (125 mP) was much smaller than that of the preferred (standard) CHAPS-containing BtLBD assay (225 mP), but nevertheless the CHAPS-free assay still ranked the competitors correctly in terms of potency. When compared with the CHAPS-free variant, however, the standard CHAPS-containing BtLBD assay was seen to require smaller amounts of BtLBD and to provide better-shaped (i.e. sigmoid) titration curves that gave Kᵢ values closer to absolute values to those from the radioligand-based assay. Moreover, the CHAPS-containing FP assay ranked muristerone A and ponasterone A close together in terms of binding affinity, just as the conventional radioligand-based assay did, whereas the CHAPS-free FP assay exaggerated the binding affinity of ponasterone A relative to muristerone A. Overall, it was clear that the performance of BtLBD competitive inhibition assays were enhanced by the presence of 2 mM CHAPS. It is expected that performing the LcLBD and HaLBD assays in the presence of 2 mM CHAPS will also improve the performance of these assays, for example by enhancing their dynamic range. It is possible that CHAPS might also improve some aspects of MpLBD competitive inhibition assays.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Ashburner, M., Chihara, C., Meltzer, P. & Richards, G. (1974). Temporal control of puffing activity in polytene chromasomes, Cold Spring Harb. Symp. Quant. Biol. 38, 655-62.
Becker, E. (1941). Uber Versuche zur Anreicherung und physiologische Charakterisierung und Wirkstoffes der Puparislerung. Biol. Zbl. 61, 360-388.
Checovich, W. J., Bolger, R. E., & Burke, T. (1995) Fluorescence Polarization—a new tool for cell and molecular biology. Nature 375, 254-25.
Cheng, Y. C., & Prusoff, W. H. (1973) Relationship between the inhibition constant and the concentration of inhibitor which causes 50% inhibition (I₅₀) of an enzymatic reaction. Biochem. Pharmacol. 22, 3099-3108.
Cherbas, P., Cherbas, L., Lee, S. & Nakanishi, K. (1988). 26-[¹²⁵I]iodoponasterone A is a potent ecdysone and a sensitive radioligand for ecdysone receptors. Proc. Natl. Acad. Sci. USA 85, 2096-100.
Clément, C. Y., Bradbrook, D. A., Lafont, R., Dinan, L. (1993). Assesesment of a microplate-based bioassay for the detection of ecdysteroid-like or antiecdysteroid activities. Insect Biochem Mol Biol 23, 187-193.
Cymborowski, B. (1989). Bioassays for ecdysteroids. In Ecdysone: from Chemistry to Mode of Action (J. Koolman, ed.), pp. 144-149, Thierne Medical Publishers, New York.
Dhadialla, T. S., Carlson, G. R. & Le, D. P. (1998). New insecticides with ecdysteroidal and juvenile hormone activity. Annu. Rev. Entomol. 43, 545-69.
Dinan, L., Hormann, R. E., Fujimoto, T. (1999). An extensive ecdysteroid CoMFA. J. Comput. Aided Mol. Des. 13, 185-207.
Fristrom J. W. & Yund, M. A. (1976). Characteristics of the action of ecdysones on Drosophila imaginal discs cultured in vitro. In Invertebrate Tissue Culture Research Applications (K. Maramorosch (Ed.)), pp. 161-178, Academic Press, New York.
Hannan, G. N. & Hill, R. J. (1997). Cloning and characterization of LcEcR: a functional ecdysone receptor from the sheep blowfly Lucilia cuprina. Insect Biochem. Mol. Biol. 27, 479-88.
Hannan, G. N. & Hill, R. J. (2001). LcUSP, an ultraspiracle gene from the sheep blowfly, Lucilia cuprina: cDNA cloning, developmental expression of RNA and confirmation of function. Insect. Biochem. Mol. Biol. 31, 771-81.
Hill, R. J., Segraves, W. A., Choi, D., Underwood, P. A. & Macavoy, E. (1993). The reaction with polytene chromosomes of antibodies raised against Drosophila E75A protein. Insect Biochem Mol Biol 23, 99-104.
Horn, D. H. S. and Bergamasco, R. (1985). Chemistry of Ecdysteroids. in ‘Comprehensive Insect Physiology, Biochemistry and Pharmacology’, Volume 7, Endocrinology 1, (G. A. Kerkut and L. I. Gilbert eds) pp 185-248, Pergamon Press Oxford.
Hulme, E. C., & Birdsall, N. J. M. (1992) Strategy and tactics in receptor-binding studies. In Receptor-Ligand Interactions—A Practical Approach (Ed. Hulme, E. C.), pp. 63-176, IRL Press, OUP, Oxford.
Jiskoot, W., Hoogerhout, P., Beuvery, E. C., Herron, J. N., & Crommelin, D. J. A. (1991) Preparation and application of a fluorescein-labeled peptide for determining the affinity constant of a monoclonal antibody-hapten complex by Fluorescence Polarization. Anal. Biochem. 196, 421-426.
Koelle, M. R., Talbot, W. S., Segraves, W. A., Bender, M. T., Cherbas, P., & Hogness, D. S. (1991) The Drosophila EcR gene encodes an ecdysone receptor, a new member of the steroid receptor superfamily. Cell 67, 59-77.
Lafont, R., & Dinan, L. (2003) Practical uses for ecdysteroids in mammals including humans: an update. J. Insect Sci. 3, article 7, Online at insectscience.org/3.7
Minakuchi, C., Nakagawa, Y., & Miyagawa, H. (2003). Binding affinity of nonsteroidal ecdysone agonists against the ecdysone receptor complex determines the strength of their molting hormonal activity. Eur J Biochem. 270, 4095-104.
Oberdorster, E., Clay, M. A., Cottam, D. M., Wilmot, F. A., McLachlan, J. A. & Milner, M. J. (2001). Common phytochemicals are ecdysteroid agonists and antagonists: a possible evolutionary link between vertebrate and invertebrate steroid hormones. J. Steroid Biochem. Mol. Biol. 77, 229-238.
Odinokov, V. N., Galyautdinov, L. V., Nedopekin, D. V., Khalilov, L. M. (2003). Trifluoroacetylation and dehydration of 20-hydroxyecdysone acetonides. Synthesis of stachisterone B. Russ. Chem. Bull., Int. Ed. 52, 232-236
Oro, A. E., McKeown, M. & Evans, R. M. (1990). Relationship between the product of the Drosophila ultraspiracle locus and the vertebrate retinoid X receptor. Nature 347, 298-301.
Orwicki, J. (2000) Fluorescent Polarization and anisotropy in high throughput screening: perspectives and primer. J. Biomol. Screen. 5, 297-306.
Padidam, M., Gore, M., Lu, D. L., & Smimova, O. (2003) Chemical-inducible, ecdysone receptor-based gene expression system for plants. Transgenic Res. 12, 101-9
Parker, G. J., Law, T. L., Lenoch, F. J., & Bolger, R. E. (2000) ‘Development of high-throughput screening assays using Fluorescence Polarisation: nuclear receptor-ligand binding and kinase/phosphatase assays.’ J. Biomol. Screen. 5, 77-88.
Pis., J., Hykl, J., Budesinsky, M., Harmatha, J. et al. (1994) Regioselective synthesis of 20-hydroxyecdysone glycosides. Tetrahedron 50, 9679-9690.
Prystay, L., Gosselin, M., Banks, P. (2001) Determination of equilibrium dissociation constants in Fluorescence Polarization. J. Biomol. Screen. 6, 141-150.
Sage, B. A., Horn, D. H. S., & Landon, T. M. (1986) Alternative ligands for measurement and purification of ecdysteroid receptors in Drosophila KC cells. Arch. Insect Biochem. Physiol. Suppl. 1, 25-33.
Sportsman, J. R., & Leytes, L. J. (2000) Miniaturization of homogeneous assays using fluorescence polarization. Drug Discovery Today, HTS Supplement 1(1), 27.
Suksamrarn, A., Pattanaprateep, P. (1995) Selective acetylation of 20-hydroxyecdysone—partial synthesis of some minor ecdysteroids and analogs. Tetrahedron 51, 10633-10650.
Suksamrarn, A., Pattanaprateep P., Tanachatchairatana T., Haritakun W., Yingyongnarongkul B., Chimnoi N. (2002) Chemical modifications at the 22-hydroxyl group of ecdysteroids: alternative structural requirements for high moulting activity. Insect Biochem. Mol. Biol. 32, 193-197
Sundaram, M., Palli, S. R., Krell, P. J., Sohi, S. S., Dhadialla, T. S. & Retnakaran, A. (1998). Basis for selective action of a synthetic molting hormone agonist, RH-5992 on lepidopteran insects. Insect Biochem. Mol. Biol. 28, 693-704.
Wedin, R. (1999) One-step fluorescence HTS assays are getting faster, cheaper, smaller and more sensitive. Modern Drug Discovery 2(3), 61-71.
Williams, C. M. (1967a). The juvenile hormone II. Its role in the endocine control of molting, pupation, and adult development of the eccropia silkworm. Biol. Bull. Woods Hole 121, 572-585.
Williams, C. M. (1967b). Third-generation pesticides. Sci. Am. 217, 13-7.
Wing, K. D. (1988). RH 5849, a nonsteroidal ecdysone agonist: effects on a Drosophila cell line. Science 241, 467-9.
Wing, K. D., Slawecki, R. A. & Carlson, G. R. (1988). RH5849, a nonsteroidal ecdysone agonist, effects on larval lepidoptera. Science 241, 470-472.
Yang, G., Hannan, G. N., Lockett, T. J. & Hill, R. J. (1986). Functional transfer of an elementary ecdysone gene regulatory system to mammalian cells: transient transfection and stable cell lines. Eur. J. Entomol. 92, 379-389.
Yao, T. P., Forman, B. M., Jiang, Z., Cherbas, L., Chen, J. D., McKeown, M., Cherbas, P. & Evans, R. M. (19930. Functional ecdysone receptor is the product of EcR and Ultraspiracle genes. Nature 366, 476-479.
Yund, M. A., King, D. S. & Pristrom, J. W. (1978). Ecdysteroid receptors in imaginal discs of Drosophila melanogaster. Proc. Natl. Acad. Sci. USA 75, 6039-43.

1. An ecdysteroid derivative wherein a fluorescent moiety is attached to an ecdysteroid moiety by derivatisation of a hydroxyl group on an alkyl side chain of the ecdysteroid moiety, wherein the derivative binds to an ecdysone receptor or ligand binding domain of an ecdysone receptor.

2. The ecdysteroid derivative according to claim 1 wherein the derivative has general structure 1a, 2a, or 3a; 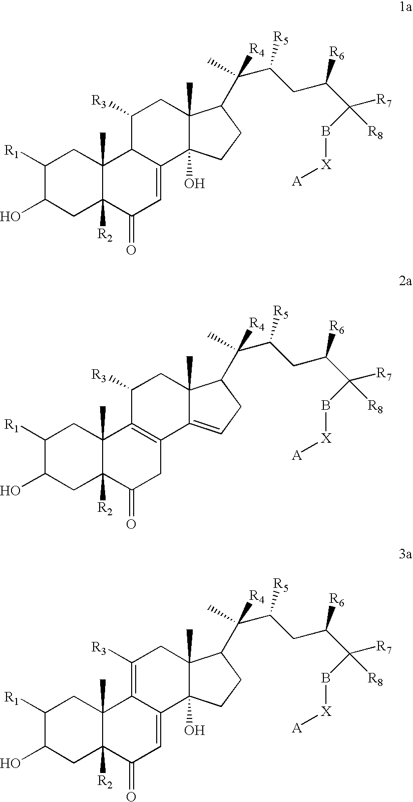
wherein B is CH₂O, CH₂S, CH₂NH, O, S, or NH; X is a linking group; A is a fluorescent moiety; R¹-R⁵ are independently selected from H, alkyl, haloalkyl, OH, or halogen, R⁷-R⁸ are independently selected from H, alkyl, haloalkyl, OH, or halogen or R⁷ and R⁸ together are ═CH₂; R⁶ is selected from H, OH, alkyl, =CH₂ or halogen, or R⁶ together with R⁷ is a double bond.

3. The derivative according to claim 2 wherein the alkyl groups are C1 to C20, more preferably, for R¹ and R² the alkyl group is C1 to C5.

4. The derivative according to claim 2 wherein R¹, and R⁵ are OH; R² is H or OH; R³ is H; R⁴ is H or OH; R⁶ is selected from H, OH, CH₃, CH₃CH₂, or (CH₃)₂ CH; R⁷ and R⁸ are independently selected from H, OH, CH₃, or R⁷ and R⁸ together can be =CH₂, R⁶ together with R⁷ can be a double bond.

5. The derivative according to claim 2 wherein X is selected from the group consisting of C(O)NH, C(S)NH, SO₂ and C(O).

6. The derivative according to claim 2 wherein B is CH₂O or O.

7. The derivative according to claim 2 wherein the fluorescent moiety is selected from the group consisting of unsubstituted and substituted fluorescein moieties, unsubstituted and substituted dansyl moieties, and unsubstituted and substituted coumarin moieties.

8. The derivative according to claim 2 wherein the derivative is of general structure 1a wherein R¹ and R⁵ are OH, R³ is H, R⁷ is CH₃, and B is CH₂O and X is selected from the group consisting of C(O)NH, C(S)NH, SO₂ and C(O).

9. The derivative according to claim 8 wherein R², R⁴, and R⁸ are independently selected from H, alkyl, OH, or halogen; R⁶ is selected from H, OH, alkyl, ═CH₂ or halogen.

10. The derivative according to claim 9 wherein R² is H, R⁴ is OH, R⁶ is H and R⁸ is H.

11. The derivative according to claim 2 wherein R¹, R⁴ and R⁵ are OH, R² is H or OH, R³ is H, R⁶ is H or CH₃, and R⁷ and R⁸ are H or CH₃.

12. The derivative according to claim 2 wherein the ecdysteroid moiety is selected from the group consisting of inokosterone, 26-hydroxyecdysone, 20,26-dihydroxyecdysone, makisterone B, amarasterone A, amarasterone B, ajugasterone B, sidasterone A, sidasterone B, 26-hydroxy-polypodine B, 20-hydroxyecdysone, makisterone A, polypodine B and rapisterone D.

13. The derivative according to claim 1 wherein the derivative is selected from the group consisting of:

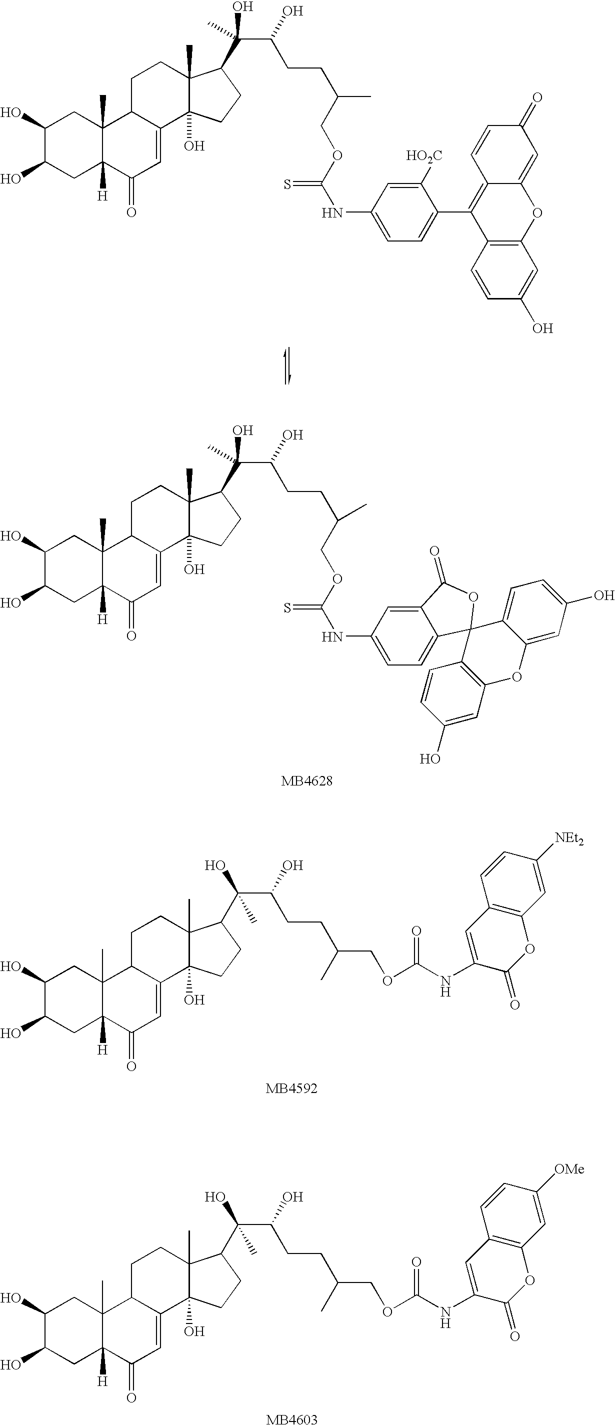

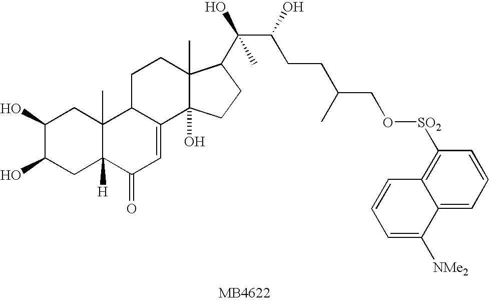

14. The ecdysteroid derivative according to claim 1 wherein the derivative has general structure 1b, 2b, or 3b; 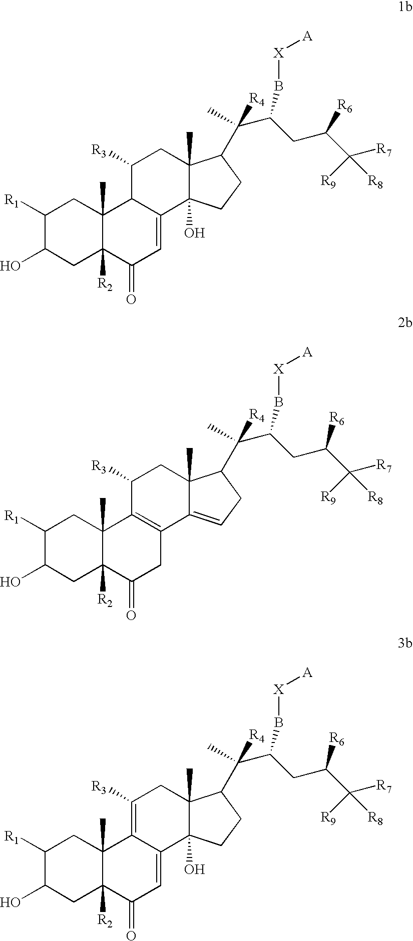
wherein B is CH₂O, CH₂S, CH₂NH, O, S, or NH; X is a linking group; A is a fluorescent moiety; R¹-R⁴, R⁷-R⁹ are independently selected from H, alkyl, haloalkyl, OH, or halogen; R⁶ is selected from H, OH, alkyl, ═CH₂ or halogen.

15. The derivative according to claim 14 wherein the alkyl groups are C1 to C20, more preferably, for R¹ and R² the alkyl group is C1 to C5.

16. The derivative according to claim 14 wherein X is selected from the group consisting of C(O)NH, C(S)NH, SO₂, and C(O).

17. The derivative according to claim 14 wherein the fluorescent moiety is selected from the group consisting of unsubstituted and substituted fluorescein moieties, unsubstituted and substituted dansyl moieties, and unsubstituted and substituted coumarin moieties.

18. The derivative according to claim 14 wherein R¹ and R⁴ are OH; R² and R³ are independently selected from H or OH; R⁶ is selected from H or CH₃, and R⁷, R⁸ and R⁹ are independently selected from the group H, OH, CH₃, F, and I.

19. The derivative according to claim 14 wherein the ecdysteroid moiety is selected from the group consisting of ponasterone A, 20-hydroxyecdysone, muristerone A, makisterone A, polypodine B, rapisterone D, 2β,3β,20R,22R-tetrahydroxy-25-fluoro-5β-cholest-8,14-dien-6-one, 5-deoxykaladasterone, 26-iodoponasterone A, and 25-fluoroponasterone A.

20. A compound selected from the group of compounds consisting of general structures 1a, 2a, and 3a which interact with an ecdysone receptor or ligand binding domain (LBD) thereof; 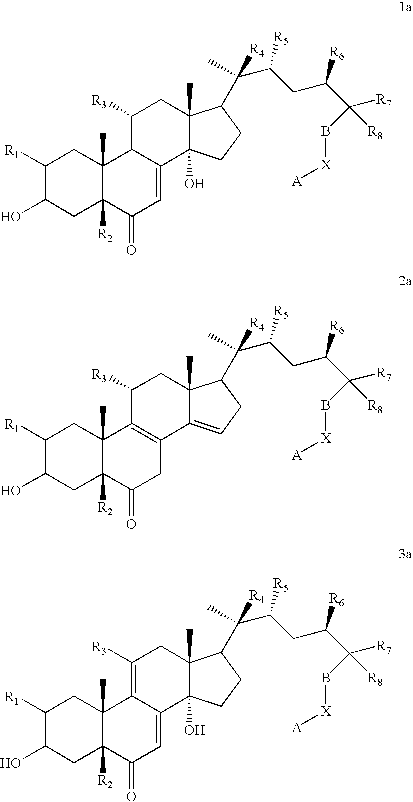
wherein B is CH₂O, CH₂S, CH₂NH, O, S, or NH; X is a linking group; A is a fluorescent moiety; R¹-R⁵ are independently selected from H, alkyl, haloalkyl, OH, or halogen, R⁷- R⁸ are independently selected from H, alkyl, haloalkyl, OH, or halogen or R⁷ and R⁸ together are ═CH₂; R⁶ is selected from H, OH, alkyl, ═CH₂ or halogen, or R⁶ together with R⁷ is a double bond.

21. The compound according to claim 20 wherein the alkyl groups are C1 to C20, more preferably, for R¹ and R² the alkyl group is C1 to C5.

22. The compound according to claim 20 wherein R¹, and R⁵ are OH; R² is H or OH; R³ is H; R⁴ is H or OH; R⁶ is selected from H, OH, CH₃, CH₃CH₂ or (CH₃)₂ CH; R⁷ and R⁸ are independently selected from H, OH, CH₃, or R⁷ and R⁸ together can be ═CH₂, R⁶ together with R⁷ can be a double bond.

23. The compound according to claim 20 wherein X is selected from the group consisting of C(O)NH, C(S)NH, SO₂ and C(O).

24. The compound according to claim 20 wherein B is CH₂O or O.

25. The compound according to claim 20 wherein the fluorescent moiety is selected from the group consisting of unsubstituted and substituted fluorescein moieties, unsubstituted and substituted dansyl moieties, and unsubstituted and substituted coumarin moieties.

26. The compound according to claim 20 wherein the compound is of general structure 1a wherein R¹ and R⁵ are OH, R³ is H, R⁷ is CH₃, and B is CH₂O and X is selected from the group consisting of C(O)NH, C(S)NH, SO₂ and C(O).

27. The compound according to claim 26 wherein R², R⁴, and R⁸ are independently selected from H, alkyl, OH, or halogen; R⁶ is selected from H, OH, alkyl, ═CH₂ or halogen.

28. The compound according to claim 27 wherein R² is H, R⁴ is OH, R⁶ is H and R⁸ is H.

29. The compound according to claim 20 wherein R¹, R⁴ and R⁵ are OH, R² is H or OH, R³ is H, R⁶ is H or CH₃, and R⁷ and R⁸ are CH₃.

30. The compound according to claim 20 wherein a fluorescent moiety is attached by derivatisation to an ecdysteroid selected from the group consisting of inokosterone, 26-hydroxyecdysone, 20,26-dihydroxyecdysone, makisterone B, amarasterone A, amarasterone B, ajugasterone B, sidasterone A, sidasterone B, 26-hydroxy-polypodine B, 20-hydroxyecdysone, makisterone A, polypodine B and rapisterone D.

31. The compound according to claim 20 wherein the compound is selected from the group consisting of:

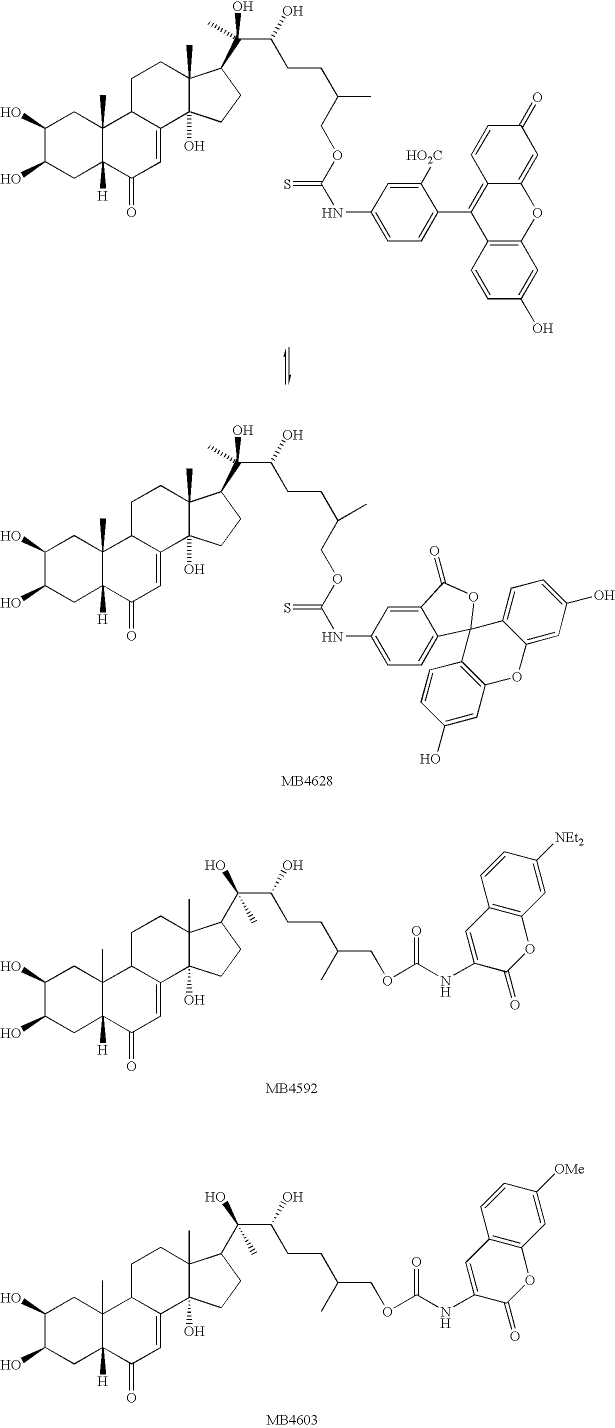

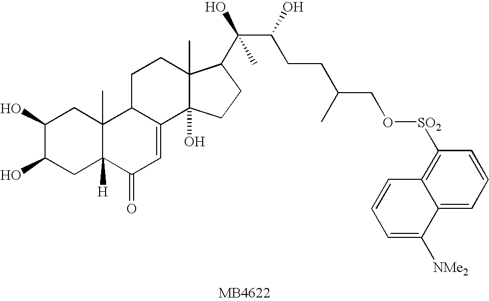

32. A compound selected from the group of compounds consisting of general structures 1b, 2b, and 3b which interact with an ecdysone receptor or ligand binding domain (LBD) thereof; 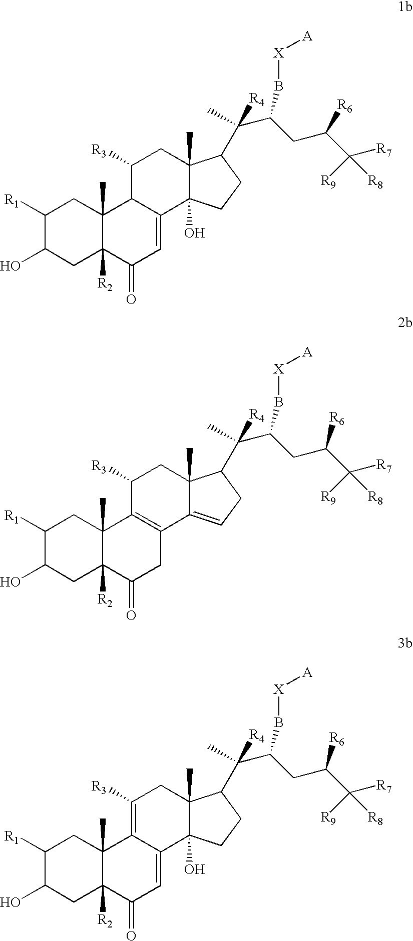
wherein B is CH₂O, CH₂S, CH₂NH, O, S, or NH; X is a linking group; A is a fluorescent moiety; R¹-R⁴, R⁷-R⁹ are independently selected from H, alkyl, haloalkyl, OH, or halogen; R⁶ is selected from H, OH, alkyl, ═CH₂ or halogen.

33. The compound according to claim 32 wherein the alkyl groups are C1 to C20, more preferably, for R¹ and R² the alkyl group is C1 to C5.

34. The compound according to claim 32 wherein X is selected from the group consisting of C(O)NH, C(S)NH, SO₂, and C(O).

35. The compound according to claim 32 wherein the fluorescent moiety is selected from the group consisting of unsubstituted and substituted fluorescein moieties, unsubstituted and substituted dansyl moieties, and unsubstituted and substituted coumarin moieties.

36. The compound according to claim 32 wherein R¹ and R⁴ are OH; R² and R³ are independently selected from H or OH; R⁶ is selected from H or CH₃, and R⁷, R⁸ and R⁹ are independently selected from the group H, OH, CH₃, F, and I.

37. The compound according to claim 32 wherein the fluorescent moiety is attached by derivatisation to an ecdysteroid is selected from the group consisting of ponasterone A, 20-hydroxyecdysone, muristerone A, makisterone A, polypodine B, rapisterone D, 2β,3β,20R,22R-tetrahydroxy-25-fluoro-5β-cholest-8,14-dien-6-one, 5-deoxykaladasterone, 26-iodoponasterone A, and 25-fluoroponasterone A.

38. A compound which is an ecdysteroid mimic wherein the compound comprises a non-ecdysteroid moiety that interacts with an ecdysone receptor or ligand binding domain thereof, and wherein the compound further comprises a fluorescent moiety.

39. The compound according to claim 38 wherein the compound comprises a substituted or unsubstituted dibenzoyl hydrazine moiety that interacts with an ecdysone receptor or ligand binding domain thereof, and wherein the compound further comprises a fluorescent moiety attached through a phenyl ring substitutent or a nitrogen atom in the dibenzoyl hydrazine moiety.

40. A method for screening a candidate compound for its ability to interact with an ecdysone receptor or ligand binding domain (LBD) thereof in a competitive inhibition format, the method comprising the steps of:
(a) incubating with an ecdysone receptor or LBD thereof, a candidate compound and the derivative according to claim 1; and
(b) measuring the extent of binding of the derivative according to claim 1 to the ecdysone receptor or LBD thereof.

41. A method for screening a candidate compound for its ability to interact with an ecdysone receptor or ligand binding domain (LBD) thereof in a competitive inhibition format, the method comprising the steps of:
(a) incubating with an ecdysone receptor or LBD thereof, a candidate compound and the compound according to claim 20; and
(b) measuring the extent of binding of the compound according to claim 20 to the ecdysone receptor or LBD thereof.

42. A method for screening a candidate compound for its ability to interact with an ecdysone receptor or ligand binding domain (LBD) thereof in a competitive inhibition format, the method comprising the steps of:
(a) incubating with an ecdysone receptor or LBD thereof, a candidate compound and the derivative according to claim 38; and
(b) measuring the extent of binding of the derivative according to claim 38 to the ecdysone receptor or LBD thereof.

43. The method according to claim 40, wherein the competitive inhibition format is a fluorescence polarization assay.

44. The method according to claim 40, wherein the assay is conducted in a microtitre plate well.

45. An insecticidal compound identified by the assay according to claim 40.

46. An effector compound for ecdysone receptor gene switches, the compound identified by the assay according to claim 40.
